(19) United States
(12) Patent Application Publication
(10) Pub. No.: US 2006/0275816 A1
(43) Pub. Date: Dec. 7, 2006

(54) Methods for identifying drug pharmacology and toxicology

(75) Inventors: Barry Steven Henderson, Hillsborough, NC (US); Richard Bentley Cheatham, Durham, NC (US)

Correspondence Address: SULLIVAN & WORCESTER LLP, ONE POST OFFICE SQUARE, BOSTON, MA, 02109, US

(73) Assignee: Ribonomics, Inc., Durham, NC (US)

(21) Appl. No.: 11/446,864

(22) Filed: Jun. 5, 2006

Related U.S. Application Data
(60) Provisional application No. 60/687,966, filed on Jun. 7, 2005.

(51) Int. Cl. C12Q 1/68

(52) U.S. Cl. 435/6

(57) ABSTRACT

The invention combines a microarray and cell-based screening strategy that enables rapid identification of possible mechanisms underpinning the pharmacology and toxicology of drug candidates. The methods of the invention identified unique properties relating to apoptosis and the anti-inflammatory response elicited by several peroxisome proliferator activated receptor gamma (PPARγ) ligands. The methods illustrate, for example, that PPARγ ligands that are safe and effective drugs (e.g., Actos, Avandia) either do not induce apoptosis or only modestly induce apoptosis. Conversely, PPARγ ligands that have failed clinical development (e.g., Ciglitazone; Day, C., Diabet. Med., 16: 179-192 (1999)) or that have been withdrawn from the market (e.g., Troglitazone (Rezulin)) due to hepatotoxicity are potent inducers of apoptosis. The methods of the invention also illustrate that suppression of gene expression and protein expression for several pro-inflammatory factors by some PPARγ ligands occurs as a consequence of apoptotic induction (i.e., apoptosis produces an anti-inflammatory response). The invention also provides biomarkers for cellular pathways and methods for stratifying patient groups according to their biomarker expression as well as biomarkers that discriminate safe and effective drugs from compounds that have acute toxicities. These biomarkers provide novel insights into the mechanism of action and toxicity for test compounds, including cell death, anti-inflammatory activity, hepatotoxicity, and carcinogenicity. The methods are highly scalable and have broad application from discovery to the clinic, including compound prioritization, predictive pharmacology and toxicology; mechanism of action studies; and prognostic and diagnostic biomarker discovery.

### CROSS-REFERENCE TO RELATED APPLICATION

This application claims priority to U.S. Provisional Patent Application No. 60/687,966, filed on Jun. 7, 2005 the entire contents of which is incorporated by reference.

### GOVERNMENT LICENSE RIGHTS

The U.S. Government has a paid-up license in this invention and the right in limited circumstances to require the patent owner to license others on reasonable terms as provided for by the terms of DAMD17-03-1-0516 awarded by The Department of Defense(DOD) Breast Cancer Research Program.

### FIELD OF THE INVENTION

The invention relates to ex vivo methods for identifying or predicting drug pharmacology and toxicology in vivo and for identifying biomarkers using a microarray and/or cell-based assay.

### BACKGROUND OF THE INVENTION

Preclinical testing of drug pharmacology and toxicology is generally based on the results from a series of biochemical, cellular and animal studies that together are used to select the most promising drug candidates for development. While some of these screens are reused for many therapeutic programs (e.g., mouse toxicity, p450 assays) others assess specific biological endpoints that are not portable outside of a specific therapeutic area (e.g., insulin secretion from pancreatic beta cells). These studies can take years to complete at significant cost to the industry and are often poor indicators of the actual efficacy and safety of drugs in humans.

One of the most significant problems in drug discovery and development is the attrition of compounds. Currently, 80% of compounds entering Phase 3 trials survive to become a marketed drug. The attrition rates in earlier stages of development are significantly worse, leading to fewer than 1 in 10,000 early stage candidates making it to market. Estimates are that the development cost of every drug includes ˜$70 million US dollars for candidates that fail to make it to market. Moreover, reducing attrition by a single percentage point or enabling compounds destined to fail to be eliminated from development earlier are estimated to lead to savings in excess of hundreds of millions of US dollars in development costs for a given drug. Consequently, there is significant interest in the pharmaceutical industry for technologies that will allow companies to predict which compounds are likely to be the most safe and effective.

As the pharmaceutical industry has struggled to increase the efficiency of their drug pipelines, a number of new approaches to assess the pharmacology and toxicology of drug candidates have emerged and have been incorporated into development programs. Computer programs have been developed that predict drug candidate properties, including toxicity and pharmacology, by comparing structural features and physical properties of test compounds to databases containing known compounds. Although these methods can be applied economically to large numbers of compounds and are useful components of an overall candidate evaluation strategy, the predictions must be borne out in experimentation. There has also been adaptation of specific biochemical and cell based assays to address general pharmacological and toxicological properties (e.g., p450 assays, hepatic enzyme activation, etc.). However many of these assays have proven difficult to scale and therefore have limited scope in terms of the number of compounds that can be assessed.

In addition, numerous commercial efforts to leverage gene expression analysis for predictive pharmacology and toxicology, as well as biomarker discovery have emerged in recent years. In general, these programs typically involve the use of commercial large scale or whole genome microarrays coupled to compound testing in animal models. Two major areas of application for gene expression microarrays are (1) pharmacogenomics, the use of gene expression technologies to delineate the inherited factors influencing drug concentrations and/or effects among individuals or populations and (2) toxicogenomics, the use of gene expression technologies to identify responses to toxicant exposure and variation in population response. In practice, these classifications represent a continuum of applications whose main goals are to identify the right medicine for the right patient: personalized medicines. Although there has been significant interest in these applications since microarrays first appeared in the mid 1990s, clinical applications are only beginning to emerge. In the last few years several examples of personalized medicines have been approved for sale, including Herceptin (Genentech1; trastuzumab) for treatment of breast tumors overexpressing HER2, Gleevec (Novartis; imatinib) for lymphoma, and Erbitux (ImClone Systems, Bristol-Myers Squibb and Merck KGaA; cetuximab), a colorectal cancer treatment. Each of these represents examples in which development was based at least in part on biomarkers identified by gene expression analysis or that rely on pharmacogenomic testing to identify responsive patients. Iconix Pharmaceuticals, Inc., Icoria, Inc., Gene Logic, Inc., and Curagen Corp. are also major competitors in this arena. Each has developed toxicogenomic offerings based on screening known drugs and toxins in animals and coupling that information with traditional histopathological analysis to identify biomarkers of specific toxicity and efficacy. Icoria's business couples whole genome expression analysis with metabolic profiling to identify predictive markers as well as straight forward toxicogenomic screening through an interaction with the National Institutes of Environmental Health Science (NIEHS). Of relevance to this application and these efforts are U.S. Pat. Nos. 6,801,859, 6,635,423 and 6,852,845. Patient stratification based on genetic variation analysis is now becoming part of clinical trial design and treatment choice, especially with respect to variations in drug metabolism enzymes. Although these examples provide some useful information, biomarker discovery, patient stratification and development of personalized medicines is still in its infancy.

Commercial microarray platforms continue to press for comprehensive gene content. For example, Affymetrix offers the Human Genome U133 microarrays that enable detection of over 47,000 human transcripts and Agilent supplies a Whole Human Genome Microarray for detection of approximately 41,000 mRNAs. These tools have many potential applications, particularly in discovery research for identifying new genes and gene products involved in biological processes and disease states. However, the datasets generated using these tools are extremely large making them difficult to manage and analyze. Adding to these challenges, microarray data sets from large survey arrays such as these have proven to be extremely noisy and poorly reproducible. This makes detection of low abundance transcripts and detection of modest, but biologically significant changes in gene expression extremely challenging using these tools. Importantly, many regulatory molecules, including certain transcription factors, are expressed at low levels and modest changes in their expression level can signal or result in significant biological consequences. These factors combine to make elucidation of biological mechanisms extremely challenging using existing tools.

Toxicology-specific microarrays have also been developed. Many of these products are simply large scale or whole genome mouse or rat microarrays, which are the most common model systems used to evaluate drug toxicity in preclinical development. Although these tools are attractive complements to traditional toxicology studies, they suffer the same limitations due to size as the human whole genome arrays. Moreover, even though the rat and mouse have been studied extensively, the gene sequence databases and annotation data lag considerably behind that for human genes, making mechanistic studies difficult. A second class of toxicology arrays that have appeared contain features for known toxicology markers, such as the National Institutes of Health ToxChip or the Oligo GEArray® Mouse Toxicology & Drug Resistance Microarray (OMM-401). These microarrays are significantly smaller than the whole genome mouse and rat arrays (6700 and 263 genes, respectively). These tools avoid problems of large scale data sets, but are of little, if any, use for elucidating mechanisms.

Smaller, focused microarrays have appeared for investigation of specific biological processes, states or pathways. For example, microarrays focused on cell cycle, inflammatory response, signal transduction, transcription factors, cytochromes, cancer, or development can be obtained commercially. These tools enable researchers to explore a particular biological state or process in depth without being overwhelmed and distracted by other changes that may be occurring. However, the scope of biological pathways and processes that these tools can survey is likely to be too limiting to be broadly useful for investigating the mechanisms of drug pharmacology and toxicology.

Certain chemical compounds in the thiazolidinediones (TZDs) family have demonstrated problematic toxicity that has had a significant negative impact on their development as thereapeutics. TZDs target peroxisome proliferator activated receptors, members of the nuclear receptor (NR) superfamily of ligand activated transcription factors which includes peroxisome proliferator activated receptor alpha (PPARα), peroxisome proliferator activated receptor gamma (PPARγ), and peroxisome proliferator activated receptor delta (PPARδ). Nuclear receptors exert their biological effects by activating or suppressing suppression of specific subsets of genes in response to hormone or ligand binding. Ligand binding induces conformational changes leading to dissociation of corepressor (N—CoR) proteins and association with (tissue) specific coactivator (N—CoR) proteins. The constellation of genes that are expressed in response to ligand binding is determined through ligand-induced conformational changes that dictate the N—CoR/N—CoA interaction pattern and, therefore, the promoter sequence(s) to which the receptor/transcription factor binds.

PPARα and PPARγ work together in the maintenance of energy homeostasis. Activation of PPARδ leads to expression of genes involved in lipid catabolism, a property that has been exploited by drugs used in the treatment of hyperlipidaemae, including Clofibrate, Fenofibrate, and Gemfibrozil. PPARγ is involved in maturation (differentiation) of adipocytes and expression of genes involved in lipogenesis. PPARγ is also an important factor in regulating the body's ability to utilize insulin and several drugs that target PPARγ, including Actos® (Pioglitazone, Takeda) and Avandiag® Rosiglitazone, GlaxoSmithKline), are currently marketed for the treatment of type 2 diabetes. These two PPARγ ligand drugs account for over $3 billion US dollars in annual world wide sales. There are currently forty three PPAR research and development programs in existence world wide with 12 PPARγ ligands currently in various stages of clinical development. The ability to determine if these agents exhibit toxicity earlier in the development cycle could lead to significant cost savings and could enable better and safer candidates to be advanced sooner.

PPARγ agonists are also being investigated for utility in several other therapeutic areas including cancer (antiproliferative and antiangiogenic activities), such as colon cancer, pancreatic cancer, and breast cancer (Demetri, G. D., et al., Proc. Natl. Acad. Sci. USA, 96: 3951-3956 (1999); Tanaka, T., et al., Cancer Res., 61: 2424-2428 (2001); Gupta, R. A., et al., J. Biol. Chem., 278: 7431-7438 (2003); Gupta, R. A., et al., J. Biol. Chem., 276: 29681-29687 (2001); Kawa, S., et al., Pancreas, 24: 1-7 (2002); Elstner, E., et al., Proc. Natl. Acad. Sci. USA, 95: 8806-8811 (1998); Clay, C. E., et al., Carcinogenesis, 20: 1905-1911 (1999); Kumagai, T., et al., Clin. Cancer Res., 10: 1508-1520 (2004); Koga, H., et al., Hepatology, 33: 1087-1097 (2001); Yoshizawa, K., et al., Cancer, 95: 2243-2251 (2002); Shimada, T., et al., Gut, 50: 658-664 (2002); Kim, E. J., et al., J. Pharmacol. Exp. Ther., 307: 505-517 (2003); Lloyd, S., et al., Chem. Biol. Interact., 142: 57-71 (2002); Toyoda, M., et al., Gut, 50: 563-567 (2002)), inflammation (Su, C. G., et al., J. Clin. Invest., 104: 383-389 (1999); Nakajima, A., et al., Gastroenterology, 120: 460-469 (2001); Kawahito, Y., et al., J. Clin. Invest., 106: 189-197 (2000); Pershadsingh, H. A., et al., J. Neuroinflammation, 1: 3 (2004); Abdelrahman, M., et al., Cardiovasc. Res., 65: 772-781 (2005)), arthritis (Kawahito, Y., et al., J. Clin. Invest., 106: 189-197 (2000)), cardiovascular disease including lipid modification and arteriosclerosis (Duval, C., et al., Trends Mol. Med., 8: 422-430 (2002); Ishibashi, M., et al., Hypertension, 40: 687-693 (2002); Fukunaga, Y., et al., Atherosclerosis, 158: 113-119 (2001); Sidhu, J. S., et al., J. Am. Coll. Cardiol., 42: 1757-63 (2003)); and polycystic ovarian syndrome (PCOS) (Mitwally, M. F., et al., J. Soc. Gynecol. Investig., 9: 163-167 (2002);Paradisi, G., et al., J. Clin. Endocrinol. Metab., 88: 576-580 (2003); Gasic, S., et al., Endocrinology, 139: 4962-4966 (1998); Veldhuis, J. D., et al., J. Clin. Endocrinol. Metab., 87: 1129-1133 (2002); Schoppee, P. D., et al., Biol. Reprod., 66: 190-198 (2002)).

Although many genes associated with the activities of PPARα and PPARγ are known, the mechanisms by which these receptors exert their biological effects are poorly understood. In addition, drugs acting through each of these receptors have significant side effects. Fibrates that act via PPARα are limited in use due to Rhabdomyolysis, which can lead to cardiac arrest and renal failure in acute cases (Muscari, A., et al., Cardiology, 97: 115-121 (2002)). The first PPARγ agonists introduced for treatment of diabetes, Trogilitazone, was withdrawn from the market and Ciglitazone was dropped from development due to hepatotoxicity (Lebovitz, H. E., Diabetes Metab. Res. Rev., 18 Suppl 2: S23-S29 (2002)). A second complication associated with all TZDs is moderate to severe peripheral, pulmonary or generalized edema. Approximately 10% of patients receiving TZD monotherapy develop edema. The percentage of patients experiencing edema increases to approximately 15% when TZDs are administered in combination with insulin (Lebovitz, H. E., Diabetes Metab. Res. Rev., 18 Suppl 2: S23-S29 (2002); Nesto, R. W., et al., Diabetes Care, 27: 256-263 (2004); Niemeyer, N. V. and L. M. Janney, Pharmacotherapy, 22: 924-929 (2002); Cheng, A. Y. and I. G. Fantus, Ann. Pharmacother., 38: 817-820 (2004)). TZD treatment is generally discontinued in diabetic patients that display edema due to the increased risk for cardiovascular disease in these patients and the concern of edema as a harbinger or sign of congestive heart failure. The most recent concern about insulin sensitizer safety arose in June of 2004 when the FDA notified all entities with ongoing clinical trials involving compounds affecting PPARγ that a two year animal toxicity study would be required before human trials lasting longer than 6 months (Jeri El-Hage, P. D., Preclinical and Clinical Safety Assessments for PPAR Agonists. 2004, US FDA). This advisory was prompted from a review of animal toxicity data (Herman, J. R., et al., Toxicol. Sci., 68: 226-236 (2002)) that revealed broad carcinogenic potential for PPARγ agonists that correlated with potency and receptor tissue distribution. Insights into the mechanistic underpinnings of the efficacy and toxicity of PPARα and PPARγ agents would provide new opportunities for development of better and safer drugs and for pharmacogenomic screens to stratify responsive patient groups.

Extensive effort has gone into the study of TZDs and pharmaceutical companies continue to pursue new and improved insulin sensitizers that target PPARγ. However, mechanisms underpinning the pharmacological benefits and the toxic side effects of PPARγ agonists are poorly understood. This makes development of new PPARγ agonists an especially high risk endeavor and the pharmacology and toxicology of these agents are not well understood until they have been evaluated in thousands of human subjects. The only alternatives for PPAR toxicity biomarkers to our knowledge are rattus genes discussed in U.S. Pat. No. 6,852,845.

A need therefore exists for a safe, efficient, ex vivo, means for determining the pharmacology and toxicity of drugs and biomarkers therefore, for example, drugs that target PPARs.

### SUMMARY OF THE INVENTION

The invention provides a ex vivo methods and compositions for identifying mechanistic biomarkers and for elucidating potential toxicity and pharmacology of chemical compounds and their underlying mechanisms and pathways. The methods of the invention provide a means for separating and characterizing the pharmacology and toxicity of drug candidates, for example, thiazolidinediones (TZDs), and provide specific screens and biomarkers that allow for population or patient stratification. The methods of the invention thus have significant utility across the drug discovery and development process. In an embodiment, the methods combine a microarray with a cell-based screen of test compounds. The gene content of the microarray focuses on regulators of human gene expression, including regulators of mRNA production (transcription), regulators of mRNA utilization (post-transcriptional regulation), as well as modulators of pathways important in the pharmacology and toxicity of drugs, for example, drugs acting via ligand activated nuclear hormone receptors. Many of these regulator or modulator genes and their encoded RNAs and proteins represent cellular “master switches”, such that changes in the abundance of their RNA transcripts and encoded proteins frequently signal or result in specific downstream biological changes or responses. Changes in the expression of these genes are therefore used as “sentinels” to indicate changes in the associated biological pathways or processes and the potential pharmacological or toxicological effects of the test chemicals (FIG. 1). The methods of the invention are an improvement over time consuming and expensive animal models, which have proven to be poor predictors of efficacy and toxicity.

In one aspect, the methods and compositions of the invention provide ex vivo methods for predicting and/or determining a certain pharmacological and/or toxicological effect of a compound in vivo. The method comprises (a) treating a cell with a compound; (b) preparing RNA from the treated cell; (c) hybridizing the RNA to a microarray comprising or consisting essentially of a plurality of nucleic acids that encode regulators of gene expression and modulators of biological pathways and/or processes involved in pharmacology and toxicology; and (d) identifying altered gene expression of the regulators and/or modulators. Altered gene expression is indicative that administration of the compound will have a certain pharmacological and/or toxicological effect in vivo. In an embodiment, the compound is a receptor ligand such as a PPAR ligand.

In another aspect, the methods and compositions of the invention provide ex vivo methods for identifying a safe drug candidate. In this embodiment, the methods and compositions of the invention further comprise the step of (e) determining the ability of the compound to induce cell death (e.g., apoptosis, necrosis, etc.) in a cell.

In another aspect, the methods and compositions of the invention provide ex vivo methods for identifying one or more biomarkers for an altered biological pathway(s) and/or process(es) in a cell that has been treated with a compound. The method comprises the steps of (a) treating a cell with a compound; (b) preparing RNA from the treated cell; (c) hybridizing the RNA to a microarray comprising or consisting essentially of a plurality of nucleic acids that encode regulators of gene expression and modulators of biological pathways and processes; and (d) identifying altered gene expression of the regulators and/or modulators, wherein the regulators and/or modulators with altered gene expression are biomarkers for an altered biological pathway(s) and/or process(es) that involves the regulators and/or modulators.

In a particular embodiment, the methods and compositions of the invention provide ex vivo methods for identifying one or more biomarkers indicative of a certain effect, such as a toxic effect, of a compound. The method comprises the steps of (a) treating a cell with a compound that has a certain effect; (b) preparing RNA from the cell; (c) hybridizing the RNA to a microarray comprising a plurality of nucleic acids that encode regulators of gene expression and modulators of biological pathways and/or processes involved in the effect (e.g., toxicity); and (d) identifying altered gene expression of the regulators and/or modulators, wherein the altered gene expression is indicative of a certain (e.g., toxic) effect of the compound in vivo.

In another aspect, the methods and compositions of the invention provide ex vivo methods for identifying a biological pathway(s) and/or process(es) that is altered in response to treating a cell with a compound. The method comprising the steps of (a) treating a cell with a compound; (b) preparing RNA from the treated cell; (c) hybridizing the RNA to a microarray comprising a plurality of nucleic acids that encode regulators of gene expression and modulators of biological pathways and/or processes; and (d) identifying altered gene expression of the regulators and/or modulators, wherein the altered gene expression is indicative that the compound acts via the biological pathway(s) and/or process(es) that involves the regulators and/or modulators.

In yet another aspect, the methods and compositions of the invention provide ex vivo methods for identifying a functional relationship between at least two biological pathways and/or processes in a cell in response to treatment with a compound. The method comprising the steps of (a) treating a cell with a compound; (b) preparing RNA from the treated cell; (c) hybridizing the RNA to a microarray comprising a plurality of nucleic acids that encode regulators of gene expression and modulators of biological pathways and/or processes; and (d) identifying altered gene expression of the regulators and/or modulators, wherein the altered gene expression of regulators and/or modulators that participate in different biological pathways and/or processes is indicative that there is a functional relationship between the biological pathways and/or processes in response to the compound. In a particular embodiment, the method identifies functional relationships between a cell death (e.g., apoptotic or necrotic) pathway or process and an NFκB pathway or process. In another embodiment, the methods identify a functional relationship between a cell death pathway or process and an inflammatory response pathway or process. In another embodiment, the methods and compositions of the invention uncouple the effects of a compound on two or more pathways or processes, such as, for example, an efficacy pathway and a toxicity pathway, such as, for example a PPAR efficacy pathway and a PPAR toxicity pathway. In another embodiment, the methods may detect the inhibition of NFκB as a consequence of PPAR induced apoptosis.

In another embodiment, the methods detect altered gene expression that is indicative of a mechanism of action of a compound, for example, a safe and effective anti-inflammatory mechanism associated with a PPAR ligand. In another embodiment, the altered gene expression is indicative of the safety of a therapeutic treatment comprising the compound or is indicative of the carcinogenicity of the compound.

In yet another embodiment, the altered gene expression of regulators or modulators can be used for grouping or stratifying a patient population in response to a compound. In a certain embodiment, that patient population is participating in a clinical trial.

The methods of the invention may further comprise the step of comparing the altered gene expression of the regulators and/or the modulators in response to the compound to the altered gene expression caused by a treatment with another compound. In another embodiment, the methods further comprise the step of determining the level of cell death in response to treatment with the compound. For example, the methods may further comprise the step of determining the level of apoptosis in the treated cell.

The biological pathway and/or process may be a cellular pathway or process, a physiological pathway or process, a biochemical pathway or process, a metabolic pathway or process, and a signaling pathway or process. In an embodiment, the pathway is a cell death pathway. In certain embodiments, the pathways or processes of the invention include nuclear receptor activation, NFκB activation, cell growth, cell proliferation, cell development, cell differentiation, apoptosis, stress, inflammation, angiogenesis, trafficking, macromolecular metabolism, RNA splicing, mRNA metabolism, transcription, translation, protein folding, exocytosis, multidrug resistance, respiration, glucose metabolism, iron homeostasis, and/or cholesterol homeostasis pathways or processes.

The regulator or modulator may be a factor that regulates transcription, a factor that regulates post-transcriptional gene expression, a factor that regulates a pharmacological pathway and/or process, and/or a factor that regulates a toxocological pathway and/or process, for example. In an embodiment, the regulator or modulator having altered gene expression is a pro-inflammatory factor or an anti-inflammatory factor. For example, the regulator or modulator having altered gene expression may be CCR2, CCL2, CCR5, CXCR4, or CXCL12.

In another embodiment, the regulator or modulator having altered gene expression is involved in apoptosis, the inflammatory response, NFκB signaling, PPAR signaling, lipid metabolism, cellular maturation or cellular differentiation (e.g., of adipocytes), lipogenesis, carcinogenicity, glucose metabolism, cell proliferation, and/or edema. In an embodiment, the altered gene expression is a biomarker for alteration in these pathways as a consequence of treatment with a compound, or provides a means for stratifying a patient population, e.g., for the predicting the efficacy or toxicity of a breast cancer treatment. The biomarkers of the invention may thus be involved in one or more of the above pathways or processes.

The pharmacological or the toxicological pathway may act at least in part via a ligand activated nuclear hormone receptor, such as a PPAR or estrogen receptor. In embodiments of the invention, the pharmacological or the toxicological pathway acts via a receptor selected from the group consisting of NR2F1, NR5A2, NR2E3, NR4A2, NR0B1, NR3C1, NR4A3, NR2C2, NR1D1, NR2F2, NR3C2, NR1I2, NR1D2, NR2C1, NR2E1, NR4A1, NR1H3, NR1H4, NR1I3, NR6A1, NR1H2, NR5A1, RARA, RARB, RARG, THRB, THRA, ESRRB, ESR2, ESRRA, ESRRG, ESRI, HNF4G, HNF4A, PPARG, PPARA, PPARD, PGR, VDR, RXRA, RXRG, RORB, RORC, RORA, GRLF1, FOXA1, and NCOA5. For example, the pharmacological or toxicological effect may be apoptosis or cell growth.

The methods and compositions of the invention are useful in testing compounds that are nuclear receptor ligands, such as an estrogen receptor ligand. For example, the estrogen receptor ligand estradiol could be tested. In another embodiment, the compound may be a peroxisome proliferator activated receptor ligand, such as a peroxisome proliferator activated receptor gamma (PPARγ) ligand, a peroxisome proliferator activated receptor alpha (PPARα) ligand, or a peroxisome proliferator activated receptor delta (PPARδ) ligand. For example, the compound may comprise Pioglitazone, Rosiglitazone, MCC-555, Troglitazone, Ciglitazone, 2-Bromohydroxydecanoic acid, Prostaglandin J2, PFOA, AV 0847, Muraglitizar (BMS,Merck), E 3030 (Eisai), LY 929 (Lilly), Ono-5129 (Ono), PLX-204 (Plexikon), Kyorin, T-131 (Amgen), Naveglitizar (Lilly), Netoglitizone (Mitsubishi), Tesaglitizar (AstraZeneca, Muraglitizar (BMS,Merck), Gemfibrozil, Fenofibrate, Clofibrate, Benzafibrate, and Wyeth 14623, or a combination thereof.

The methods and compositions of the invention are useful in detecting the toxicity to any tested cell type. In an embodiment, the methods and compositions determine hepatotoxicity of the compound. In an embodiment, gene expression of a gene that regulates cell growth, apoptosis, the inflammatory response (e.g., mediated by NFκB) is altered. In yet another embodiment, the compound is known or suspected to exert an effect on gene expression via a peroxisome proliferator activated receptor.

In embodiments of the invention, the identifying step comprises comparing gene expression of the treated cell to gene expression of a control cell (e.g., an untreated cell, a cell that is treated with a toxic compound, a cell treated with a safe drug, or a cell that is treated with a non-toxic compound). The cells used in the practice of the invention include cultured cells, for example cultured hepatic cells such as a hepatocellular carcinoma (e.g., HEPG2 cells). In other embodiments of the invention, the cell is a primary hepatocyte, a primary non-human hepatocyte, a transformed animal cell, a hepatic cell in a live animal, a pancreatic cell, a muscle cell, an adipose cell, breast cell, kidney cell, an endothelial cell, immune cell (e.g., Kupffer cell), for example.

### BRIEF DESCRIPTION OF THE DRAWINGS

The foregoing and other objects, features and advantages of the present invention, as well as the invention itself, will be more fully understood from the following description of preferred embodiments when read together with the accompanying drawings, in which:

FIG. 1 provides an illustration of the “Pathway Sentinel” strategy employed using the methods of the invention to indicate compound pharmacology and toxicology.

FIG. 2 provides an illustration of a drug discovery and development strategy employing the methods and compositions of the invention. The rows of the “predictive profiles” for FIG. 2 represent individual genes determined to be differentially expressed in HepG2 cells treated with compounds as provided herein (Table 2) relative to untreated control cells. The columns of FIG. 2 represent individual treatments (different compounds). The genes and treatments have been organized or clustered based on similarities in expression level between genes and treatments. Elevated expression relative to control is indicated by red; suppressed expression relative to control is indicated by blue; and equivelant expression relative to control is indicated by yellow. Visual inspection of these profiles indicates that each treatment elicits a unique set of changes in gene expression. However, there are common features between certain treatments as can be seen by similar patterns of red, yellow and blue across multiple treatment columns. Inspection of the similarities between treatments (e.g., genes upregulated (red) by multiple treatments in the upper left) can reveal common pharmacology and toxicology of those chemicals. Similarly, inspection of the differences between individual treatments can reveal unique pharmacological and toxicological properties of each compound.

FIG. 3 provides a graphical illustration of an experimental approach used to assess candidate compounds. (1) Cells were plated and allowed to adhere for one day. At that time various concentrations of compound were added to cells and incubated for an additional 72 hours. The concentration of compound producing 50% cell death following 72 hours of incubation with compounds was determined to be the LD₅₀ concentration. (2) Cells were plated as above and treated with compound at the predetermined LD₅₀ concentration. Following 24 hours incubation in the presence of compound, cells were collected, RNA was harvested and 3) analyzed using the RiboChip.

FIG. 4 illustrates the induction of apoptosis by TZDs. HepG2 cells were incubated for 24 hours with equimolar concentrations of the indicated TZDs in the presence or absence of the Caspase 3/7 inhibitor DEVD.

FIG. 5 illustrates the effect of TZD treatment and inhibition of apoptosis on CCR2 mRNA levels. A) Cells were treated for 24 hours at LD₅₀ concentrations of TZD with and without DEVD. B) Cells were treated for 24 hours at 175 μM TZD with and without DEVD. RNA was harvested and analyzed by QRTPCR.

FIG. 6A illustrates the effect of TZD treatment and inhibition of apoptosis on CCL2 mRNA levels. Cells were treated for 24 hours at 175 μM TZD with and without DEVD. RNA was harvested and analyzed by QRTPCR.

FIG. 6B illustrates the effect of TZD treatment and inhibition of apoptosis on CCR5 mRNA levels. Cells were treated for 24 hours at 175 μM TZD with and without DEVD. RNA was harvested and analyzed by QRTPCR.

FIG. 6C illustrates the effect of TZD treatment and inhibition of apoptosis on CXCR4 mRNA levels. Cells were treated for 24 hours at 175 μM TZD with and without DEVD. RNA was harvested and analyzed by QRTPCR.

FIG. 6D illustrates the effect of TZD treatment and inhibition of apoptosis on CXCL12 mRNA levels. Cells were treated for 24 hours at LD₅₀ concentrations of TZD with and without DEVD. RNA was harvested and analyzed by QRTPCR.

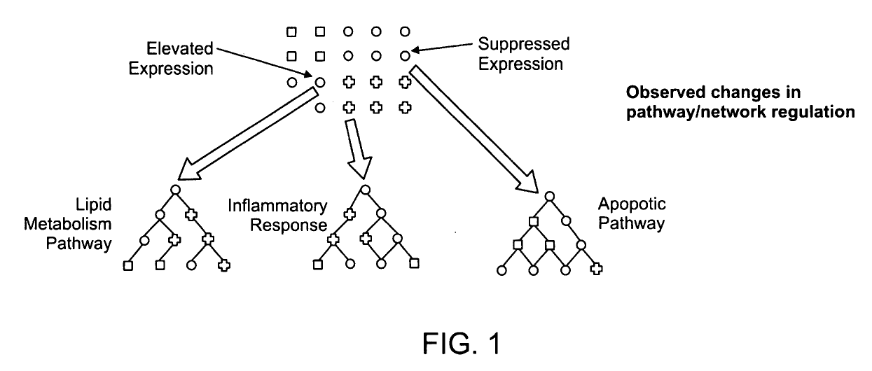

### DETAILED DESCRIPTION OF THE INVENTION

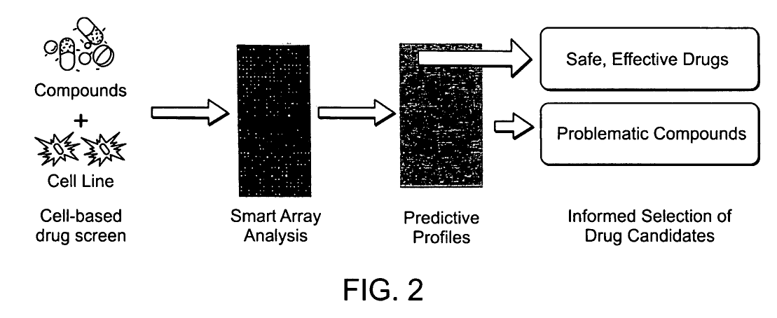

The use of the methods and compositions of the invention for drug discovery and development are illustrated in FIG. 2. The columns of the predictive profiles in FIG. 2 represent the gene expression of HEPG2 cells after treatment with a series of PPARγ and PPARα ligands, as provided herein (Table 2). The chemical profiles were grouped according to similarities in their altered gene expression. By way of illustration, the gene expression cluster in the top left of the profile comprises biomarkers for safe and effective drugs, whereas the cluster in the lower midportion of the profile comprises biomarkers for problematic compounds.

In one aspect, the cell-based screen employed in this invention is exemplified using an immortalized cell line, however the assay may be performed on any live cell, for example, a cell derived from a patient, for example a breast cancer patient, undergoing or about to undergo a drug treatment to determine the mechanisms of action and likely side effects of a drug candidate. Exemplary cells useful in the practice of the methods and compositions of the invention include hepatocytes (e.g., primary or immortalized (e.g., HepG2)); adipocytes (e.g., primary or cultured human); skeletal muscle cells; breast carcinoma cells (e.g., MCF-7); normal breast cells (e.g., tissue); cervical carcinoma cells (e.g., HeLa); colon carcinoma cells (e.g., HCT 116, LoVo); T-cells (e.g., primary or jurkat); macrophages (e.g., THP-1); monocytes (e.g., THP-1); β-cells (e.g., INS-1 cells or primary islets); neurons (e.g., primary or P-19); neuroblastoma cells (e.g., SH-SY5Y); lung carcinoma cells (e.g., A-549, NCI-H146); prostate carcinoma cells (e.g., PC3); lymphoma cells (e.g., Raji); kidney cells, and osteosarcoma cells (e.g., MG-63).

In an embodiment, the invention comprises an apoptosis assay. In other embodiments, the invention comprises an assay in addition to or instead of the apoptosis assay, which may be indicated by the results of the microarray analysis, which may indicate relationships between certain biological pathways, such as, for example an assay for cell growth, apoptosis, CYP gene/protein expression, ligand-induced global gene expression (e.g., microarray or PCR), ligand-induced target gene expression (e.g., microarray or PCR), ligand-induced alterations in expression of coactivators and corepressors (e.g., microarrays, PCR, IB), ligand-induced coactivator and/or corepressor recruitment (e.g., microarrays or in vitro), cytokine production (e.g., ELISA or IB),chemokine production (e.g., ELISA or IB), other secreted molecules such as hormones (e.g., ELISA; IB), changes in expression of cell surface proteins (e.g., markers) such as chemokine receptors and cytokine receptors (e.g., flow cytometry, FACS analysis, IB), cellular differentiation (e.g., pre-adipocyte to adipocyte or monocyte to macrophage), angiogenesis, lipolysis, glucose uptake, fatty acid synthesis serum lipids, serum free fatty acids, serum cholesterol, serum glucose, serum adiponectin, serum leptin, serum GLP-1, or a combination thereof.

In an embodiment, the methods of the invention are useful for identifying the mechanisms associated with compounds that act via any of a number of nuclear receptors, such as, for example, NR2F1, NR5A2, NR2E3, NR4A2, NR0B1, NR3C1, NR4A3, NR2C2, NR1D1, NR2F2, NR3C2, NR1I2, NR1D2, NR2C1, NR2E1, NR4A1, NR1H3, NR1H4, NR1I3, NR6A1, NR1H2, NR5A1, RARA, RARB, RARG, THRB, THRA, ESRRB, ESR2, ESRRA, ESRRG, ESR1, HNF4G, HNF4A, PPARG, PPARA, PPARD, PGR, VDR, RXRA, RXRG, RORB, RORC, RORA, GRLF1, FOXA1, and NCOA5 (Table 1).

TABLE 1

| Official | | | | |
|---|---|---|---|---|
| Symbol | Official Name | Other Aliases | Other Designations | GeneID |
| NR2F1 | nuclear receptor | HGNC: 7975, | ERBAL3, TFCOUP1; | 7025 |
| | subfamily 2, group | COUP-TFI, EAR-3, | transcription factor COUP | |
| | F, member 1 [Homo | EAR3, ERBAL3, | 1 (chicken ovalbumin | |
| | sapiens] | NR2F2, SVP44, | upstream promoter 1, v- | |
| | | TCFCOUP1, | erb-a homolog-like 3) | |
| | | TFCOUP1 | | |
| NR5A2 | nuclear receptor | HGNC: 7984, B1F, | CYP7A promoter-binding | 2494 |
| | subfamily 5, group | B1F2, CPF, FTF, | factor; b1-binding factor, | |
| | A, member 2 [Homo | FTZ-F1, FTZ- | hepatocyte transcription | |
| | sapiens] | F1beta, LRH-1, | factor which activates | |
| | | hB1F, hB1F-2 | enhancer II of hepatitis B | |
| | | | virus; fetoprotein-alpha 1 | |
| | | | (AFP) transcription | |
| | | | factor; liver receptor | |
| | | | homolog 1; nuclear | |
| | | | receptor NR5A2 | |
| NR2E3 | nuclear receptor | HGNC: 7974, | photoreceptor-specific | 10002 |
| | subfamily 2, group | ESCS, MGC49976, | nuclear receptor; retina- | |
| | E, member 3 [Homo | PNR, RNR | specific nuclear receptor | |
| | sapiens] | | | |
| NR4A2 | nuclear receptor | HGNC: 7981, HZF- | NGFI-B/nur77 beta-type | 4929 |
| | subfamily 4, group | 3, NOT, NURR1, | transcription factor | |
| | A, member 2 [Homo | RNR1, TINUR | homolog; T-cell nuclear | |
| | sapiens] | | receptor NOT; | |
| | | | intermediate-early | |
| | | | receptor protein; nur | |
| | | | related protein-1 (mouse), | |
| | | | human homolog of; | |
| | | | orphan nuclear receptor | |
| | | | NURR1; transcriptionally | |
| | | | inducible nuclear receptor | |
| | | | related 1 | |
| NR0B1 | nuclear receptor | HGNC: 7960, AHC, | gonadotropin deficiency; | 190 |
| | subfamily 0, group | AHCH, AHX, | nuclear hormone receptor | |
| | B, member 1 [Homo | DAX-1, DAX1, | | |
| | sapiens] | DSS, GTD, HHG, | | |
| | | NROB1 | | |
| NR3C1 | nuclear receptor | HGNC: 7978, GCR, | Glucocorticoid receptor, | 2908 |
| | subfamily 3, group | GR, GRL | lymphocyte; | |
| | C, member 1 | | glucocorticoid receptor | |
| | (glucocorticoid | | | |
| | receptor) [Homo | | | |
| | sapiens] | | | |
| NR4A3 | nuclear receptor | HGNC: 7982, CHN, | chondrosarcoma, | 8013 |
| | subfamily 4, group | CSMF, MINOR, | extraskeletal myxoid, | |
| | A, member 3 [Homo | NOR1, TEC | fused to EWS; mitogen | |
| | sapiens] | | induced nuclear orphan | |
| | | | receptor; neuron derived | |
| | | | orphan receptor; | |
| | | | translocated in | |
| | | | extraskeletal | |
| | | | chondrosarcoma | |
| NR2C2 | nuclear receptor | HGNC: 7972, | Nuclear hormone receptor | 7182 |
| | subfamily 2, group | TAK1, TR2R1, | TR4; TR4 nuclear | |
| | C, member 2 [Homo | TR4, hTAK1 | hormone receptor | |
| | sapiens] | | | |
| NR1D1 | nuclear receptor | HGNC: 7962, | Rev-ErbAalpha; thyroid | 9572 |
| | subfamily 1, group | EAR1, THRA1, | hormone receptor, alpha- | |
| | D, member 1 [Homo | THRAL, ear-1, | like | |
| | sapiens] | hRev | | |
| NR2F2 | nuclear receptor | HGNC: 7976, | ADP-ribosylation factor | 7026 |
| | subfamily 2, group | ARP1, COUP-TFII, | related protein 1; ARP1, | |
| | F, member 2 [Homo | COUPTFB, SVP40, | TFCOUP2; transcription | |
| | sapiens] | TFCOUP2 | factor COUP 2 (chicken | |
| | | | ovalbumin upstream | |
| | | | promoter 2, | |
| | | | apolipoprotein regulatory | |
| | | | protein) | |
| NR3C2 | nuclear receptor | HGNC: 7979, MCR, | mineralocorticoid | 4306 |
| | subfamily 3, group | MLR, MR | receptor (aldosterone | |
| | C, member 2 [Homo | | receptor) | |
| | sapiens] | | | |
| NR1I2 | nuclear receptor | HGNC: 7968, BXR, | pregnane X receptor; | 8856 |
| | subfamily 1, group I, | ONR1, PAR, | steroid and xenobiotic | |
| | member 2 [Homo | PAR1, PAR2, | receptor | |
| | sapiens] | PARq, PRR, PXR, | | |
| | | SAR, SXR | | |
| NR1D2 | nuclear receptor | HGNC: 7963, | Rev-erb-beta | 9975 |
| | subfamily 1, group | BD73, EAR-1r, | | |
| | D, member 2 [Homo | HZF2, Hs.37288, | | |
| | sapiens] | RVR | | |
| NR2C1 | nuclear receptor | HGNC: 7971, TR2, | TR2 nuclear hormone | 7181 |
| | subfamily 2, group | TR2-11 | receptor | |
| | C, member 1 [Homo | | | |
| | sapiens] | | | |
| NR2E1 | nuclear receptor | HGNC: 7973, TLL, | OTTHUMP00000040473; | 7101 |
| | subfamily 2, group | TLX, XTLL | tailless (Drosophila) | |
| | E, member 1 [Homo | | homolog; tailless | |
| | sapiens] | | homolog (Drosophila) | |
| NR4A1 | nuclear receptor | HGNC: 7980, | HMR, GFRP1; TR3 | 3164 |
| | subfamily 4, group | GFRP1, HMR, | orphan receptor; early | |
| | A, member 1 [Homo | MGC9485, N10, | response protein NAK1; | |
| | sapiens] | NAK-1, NGFIB, | growth factor-inducible | |
| | | NP10, NUR77, | nuclear protein N10; | |
| | | TR3 | hormone receptor; orphan | |
| | | | nuclear receptor HMR; | |
| | | | steroid receptor TR3 | |
| NR1H3 | nuclear receptor | HGNC: 7966, LXR- | liver X receptor, alpha | 10062 |
| | subfamily 1, group | a, LXRA, RLD-1 | | |
| | H, member 3 [Homo | | | |
| | sapiens] | | | |
| NR1H4 | nuclear receptor | HGNC: 7967, BAR, | | 9971 |
| | subfamily 1, group | FXR, HRR-1, | | |
| | H, member 4 [Homo | HRR1, RIP14 | | |
| | sapiens] | | | |
| NR1I3 | nuclear receptor | HGNC: 7969, CAR, | constitutive androstane | 9970 |
| | subfamily 1, group I, | CAR-BETA, CAR- | receptor SV1; constitutive | |
| | member 3 [Homo | SV1, CAR-SV10, | androstane receptor | |
| | sapiens] | CAR-SV12, CAR- | SV10; constitutive | |
| | | SV13, CAR-SV14, | androstane receptor | |
| | | CAR-SV21, CAR- | SV12; constitutive | |
| | | SV4, CAR-SV6, | androstane receptor | |
| | | CAR-SV7, CAR- | SV14; constitutive | |
| | | SV8, CAR-SV9, | androstane receptor SV6; | |
| | | CAR1, MB67 | constitutive androstane | |
| | | | receptor SV7; constitutive | |
| | | | androstane receptor SV9; | |
| | | | constitutive androstane | |
| | | | receptor-beta; orphan | |
| | | | nuclear hormone receptor | |
| NR6A1 | nuclear receptor | HGNC: 7985, | germ cell nuclear factor; | 2649 |
| | subfamily 6, group | GCNF, GCNF1, | retinoic acid receptor- | |
| | A, member 1 [Homo | NR61, RTR | related testis-associated | |
| | sapiens] | | receptor | |
| NR1H2 | nuclear receptor | HGNC: 7965, LXR- | LX receptor beta; liver X | 7376 |
| | subfamily 1, group | b, NER, NER-I, | receptor beta; nuclear | |
| | H, member 2 [Homo | RIP15, UNR | orphan receptor LXR- | |
| | sapiens] | | beta; oxysterols receptor | |
| | | | LXR-beta; steroid | |
| | | | hormone-nuclear receptor | |
| | | | NER; ubiquitously- | |
| | | | expressed nuclear | |
| | | | receptor | |
| NR5A1 | nuclear receptor | HGNC: 7983, | OTTHUMP00000042845; | 2516 |
| | subfamily 5, group | AD4BP, ELP, | OTTHUMP00000042846; | |
| | A, member 1 [Homo | FTZ1, FTZF1, SF- | OTTHUMP00000042847; | |
| | sapiens] | 1, SF1 | fushi tarazu factor | |
| | | | (Drosophila) homolog 1; | |
| | | | nuclear receptor AdBP4; | |
| | | | steroidogenic factor 1 | |
| RARA | retinoic acid | HGNC: 9864, | Retinoic acid receptor, | 5914 |
| | receptor, alpha | NR1B1, RAR | alpha polypeptide; | |
| | [Homo sapiens] | | nucleophosmin-retinoic | |
| | | | acid receptor alpha fusion | |
| | | | protein NPM-RAR long | |
| | | | form; nucleophosmin- | |
| | | | retinoic acid receptor | |
| | | | alpha fusion protein | |
| | | | NPM-RAR short form | |
| RARB | retinoic acid | HGNC: 9865, HAP, | HBV-activated protein; | 5915 |
| | receptor, beta | NR1B2, RRB2 | RAR, beta form; RAR- | |
| | [Homo sapiens] | | epsilon; hepatitis B virus | |
| | | | activated protein; retinoic | |
| | | | acid receptor beta 2; | |
| | | | retinoic acid receptor beta | |
| | | | 4; retinoic acid receptor | |
| | | | beta 5; retinoic acid | |
| | | | receptor, beta polypeptide | |
| RARG | retinoic acid | HGNC: 9866, | | 5916 |
| | receptor, gamma | NR1B3, RARC | | |
| | [Homo sapiens] | | | |
| THRB | thyroid hormone | HGNC: 11799, | avian erythroblastic | 7068 |
| | receptor, beta | ERBA-BETA, | leukemia viral (v-erb-a) | |
| | (erythroblastic | ERBA2, GRTH, | oncogene homolog 2; beta | |
| | leukemia viral (v- | NR1A2, THR1, | (avian erythroblastic | |
| | erb-a) oncogene | THRB1, THRB2 | leukemia viral (v-erb-a) | |
| | homolog 2, avian) | | oncogene homolog 2); | |
| | [Homo sapiens] | | generalized resistance to | |
| | | | thyroid hormone; | |
| | | | oncogene ERBA2; | |
| | | | thyroid hormone receptor | |
| | | | beta 1; thyroid hormone | |
| | | | receptor, beta; thyroid | |
| | | | hormone receptor, beta | |
| | | | (avian erythroblastic | |
| | | | leukemia viral (v-erb-a) | |
| | | | oncogene homolog 2) | |
| THRA | thyroid hormone | HGNC: 11796, | EAR-7.1/EAR-7.2; | 7067 |
| | receptor, alpha | AR7, EAR-7.1, | ERBA-related 7; THRA1, | |
| | (erythroblastic | EAR-7.2, EAR7, | THRA2, ERBA1; alpha | |
| | leukemia viral (v- | ERB-T-1, ERBA, | (avian erythroblastic | |
| | erb-a) oncogene | ERBA-ALPHA, | leukemia viral (v-erb-a) | |
| | homolog, avian) | ERBA1, | oncogene homolog); | |
| | [Homo sapiens] | MGC000261, | avian erythroblastic | |
| | | MGC43240, | leukemia viral (v-erb-a) | |
| | | NR1A1, THRA1, | oncogene homolog; | |
| | | THRA2, THRA3, | thyroid hormone receptor, | |
| | | TR-ALPHA-1, c- | alpha; thyroid hormone | |
| | | ERBA-1, c-ERBA- | receptor, alpha (avian | |
| | | ALPHA-2 | erythroblastic leukemia | |
| | | | viral (v-erb-a) oncogene | |
| | | | homolog); thyroid | |
| | | | hormone receptor, alpha | |
| | | | 1; thyroid hormone | |
| | | | receptor, alpha-2; thyroid | |
| | | | hormone receptor, alpha- | |
| | | | 3; triiodothyronine | |
| | | | receptor | |
| ESRRB | estrogen-related | HGNC: 3473, | estrogen receptor-like 2; | 2103 |
| | receptor beta [Homo | ERR2, ERRb, | nuclear receptor ERRB2; | |
| | sapiens] | ERRbeta, ERRbeta- | orphan nuclear receptor; | |
| | | 2, ESRL2, NR3B2 | steroid hormone receptor | |
| | | | ERR2 | |
| ESR2 | estrogen receptor 2 | HGNC: 3468, | estrogen receptor 2; | 2100 |
| | (ER beta) [Homo | 5p152, ER-BETA, | estrogen receptor beta | |
| | sapiens] | ESR-BETA, ESRB, | | |
| | | Erb, NR3A2 | | |
| ESRRA | estrogen-related | HGNC: 3471, | estrogen receptor-like 1 | 2101 |
| | receptor alpha | ERR1, ERRa, | | |
| | [Homo sapiens] | ERRalpha, ESRL1, | | |
| | | NR3B1 | | |
| ESRRG | estrogen-related | HGNC: 3474, | | 2104 |
| | receptor gamma | DKFZp781L1617, | | |
| | [Homo sapiens] | ERR3, NR3B3 | | |
| ESR1 | estrogen receptor 1 | HGNC: 3467, | dJ443C4.1.1 (estrogen | 2099 |
| | [Homo sapiens] | DKFZp686N23123, | receptor 1); estrogen | |
| | | ER, ESR, ESRA, | receptor 1 (alpha); | |
| | | Era, NR3A1, major | oestrogen receptor; | |
| | | ORF | steroid hormone receptor | |
| HNF4G | hepatocyte nuclear | HGNC: 5026, | | 3174 |
| | factor 4, gamma | NR2A2 | | |
| | [Homo sapiens] | | | |
| HNF4A | hepatocyte nuclear | HGNC: 5024, | HNF4-alpha; TCF14, | 3172 |
| | factor 4, alpha | FLJ39654, HNF4, | MODY, MODY1; hepatic | |
| | [Homo sapiens] | HNF4a7, HNF4a8, | nuclear factor 4 alpha; | |
| | | HNF4a9, MODY, | hepatocyte nuclear factor | |
| | | MODY1, NR2A1, | 4 alpha; transcription | |
| | | NR2A21, TCF, | factor-14 | |
| | | TCF14 | | |
| PPARG | peroxisome | HGNC: 9236, | PPAR gamma; | 5468 |
| | proliferative | HUMPPARG, | peroxisome proliferative | |
| | activated receptor, | NR1C3, PPARG1, | activated receptor gamma; | |
| | gamma [Homo | PPARG2 | peroxisome proliferator | |
| | sapiens] | | activated-receptor | |
| | | | gamma; peroxisome | |
| | | | proliferator-activated | |
| | | | receptor gamma 1; ppar | |
| | | | gamma2 | |
| PPARA | peroxisome | HGNC: 9232, | OTTHUMP00000028713; | 5465 |
| | proliferative | MGC2237, | OTTHUMP00000042872 | |
| | activated receptor, | MGC2452, NR1C1, | | |
| | alpha [Homo | PPAR, hPPAR | | |
| | sapiens] | | | |
| PPARD | peroxisome | HGNC: 9235, | nuclear hormone receptor 1 | 5467 |
| | proliferative | FAAR, MGC3931, | | |
| | activated receptor, | NR1C2, NUC1, | | |
| | delta [Homo | NUCI, NUCII, | | |
| | sapiens] | PPAR-beta, | | |
| | | PPARB | | |
| PGR | progesterone | HGNC: 8910, | | 367 |
| | receptor [Homo | NR3C3, PR | | |
| | sapiens] | | | |
| VDR | vitamin D (1,25- | HGNC: 12679, | vitamin D (1,25- | 7421 |
| | dihydroxyvitamin | NR1I1 | dihydroxyvitamin D3) | |
| | D3) receptor [Homo | | receptor | |
| | sapiens] | | | |
| RXRA | retinoid X receptor, | HGNC: 10477, | | 6257 |
| | alpha [Homo | NR2B1 | | |
| | sapiens] | | | |
| RXRG | retinoid X receptor, | HGNC: 10479, | OTTHUMP00000060418; | 6258 |
| | gamma [Homo | NR2B3, RXRC | retinoic acid receptor | |
| | sapiens] | | RXR-gamma | |
| RORB | RAR-related orphan | HGNC: 10259, | RAR-related orphan | 6096 |
| | receptor B [Homo | NR1F2, ROR- | receptor beta; nuclear | |
| | sapiens] | BETA, RZRB, | receptor RZR-beta; | |
| | | bA133M9.1 | retinoic acid-binding | |
| | | | receptor beta | |
| RORC | RAR-related orphan | HGNC: 10260, | RAR-related orphan | 6097 |
| | receptor C [Homo | NR1F3, RORG, | receptor gamma; nuclear | |
| | sapiens] | RZRG, TOR | receptor ROR-gamma; | |
| | | | retinoic acid-binding | |
| | | | receptor gamma | |
| RORA | RAR-related orphan | HGNC: 10258, | RAR-related orphan | 6095 |
| | receptor A [Homo | NR1F1, ROR1, | receptor alpha; ROR- | |
| | sapiens] | ROR2, ROR3, | alpha; retinoic acid | |
| | | RZRA | receptor-related orphan | |
| | | | receptor alpha; | |
| | | | transcription factor RZR- | |
| | | | alpha | |
| GRLF1 | glucocorticoid | HGNC: 4591, GRF- | | 2909 |
| | receptor DNA | 1, KIAA1722, | | |
| | binding factor 1 | MGC10745, P190- | | |
| | [Homo sapiens] | A, P190A, | | |
| | | p190RhoGAP | | |
| FOXA1 | forkhead box A1 | HGNC: 5021, | hepatocyte nuclear factor | 3169 |
| | [Homo sapiens] | HNF3A, | 3; hepatocyte nuclear | |
| | | MGC33105, | factor 3, alpha | |
| | | TCF3A, alpha | | |
| NCOA5 | nuclear receptor | HGNC: 15909, CIA, | coactivator independent | 57727 |
| | coactivator 5 [Homo | bA465L10.6 | of AF-2 | |
| | sapiens] | | | |

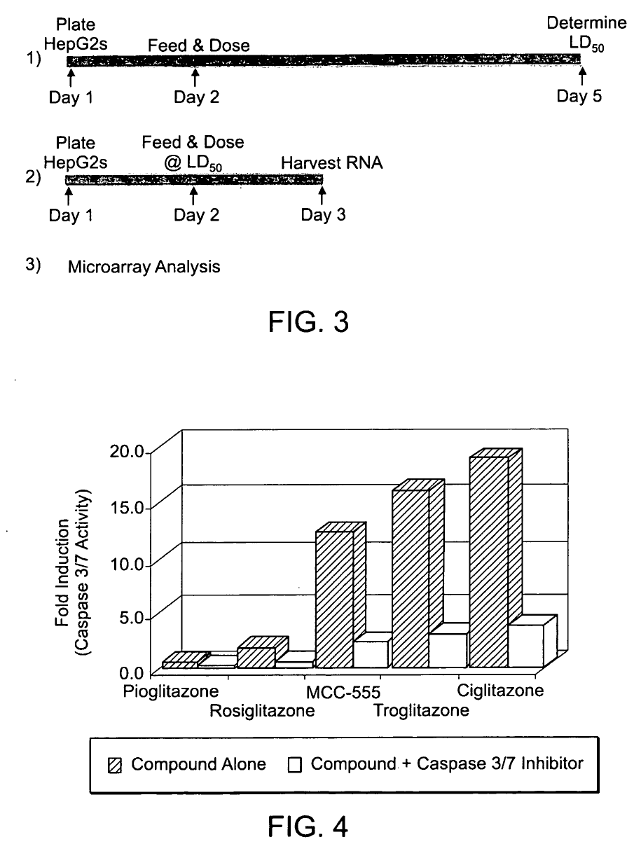

In an embodiment, human hepatocellular carcinoma HepG2 cells were used to test the effect of a compound on liver biology and toxicology. Liver is a target tissue for many compounds and is a dominant site of toxicity observed in drug development. In an embodiment, a single acute dose of a compound is used. For example, the concentration of a compound required to produce 50% cell death (LD₅₀) following 72 hours of exposure to the test agent was determined. Cells treated with the test compound at the predetermined LD₅₀ concentration were harvested after only 24 hours of exposure (FIG. 3). This is comparable to the dosing strategy used in preclinical animal studies of acute toxicity in which rodents are exposed to drug doses that lead to 50% or 90% killing over short time periods. The choice of high dose identifies the possible modes of toxicity and detects low-incidence responses. Thus, the conditions represented an acute dosing with a measurable adverse event, cell death, that when coupled with the gene content of the microarray can be used to effectively predict toxicology and pharmacology of test compounds. Thus, the methods of the invention provide a microarray-based biomarker discovery and mechanistic screening for drug pharmacology and toxicology.

In another embodiment, time and dose dependent changes in gene expression are determined in order to resolve the pharmacology and toxicology of the test agents. Earlier time points (e.g., 6 hours of compound exposure) as well as lower or higher doses can also be used to resolve pharmacological and toxicological responses. In an embodiment, the cells are treated with an LD₅₀ dose of the compound. In another embodiment, the cells are treated with a dose of the compound that is lower or higher than the LD₅₀ dose. In another embodiment, the cell is treated for about 2, about 4, about 6, about 8, about 10, about 12, about 14, about 16, about 18, about 20, about 22 hours, or about 24 hours or greater.

Thirteen (13) compounds, including six (6) ligands of PPARα and seven (7) ligands of PPARγ (Table 2) were analysed using the predictive pharmacology and toxicology platform and protocols outlined above and in the examples. A primary objective of this study was to identify biomarkers suggestive of unique pharmacology and toxicology for individual treatments that could be used to elucidate mechanistic distinctions between effective drugs and failed compounds.

TABLE 2
Compounds, Targets, Efficacy and Toxicity Properties, and LD₅₀ Concentrations
in HepG2 Cells

| Compound | Target | Properties | LD₅₀ | |
|---|---|---|---|---|
| Bezafibrate | PPARα | Agonist, Hyperlipidemea drug | 1940 | μM |
| Clofibrate | PPARα | Agonist, Hyperlipidemea drug | 240 | μM |
| Diethylhexylphthalate (DEHP) | PPARα | Agonist, Environmental contaminant with | 34 | μM |
| | | PPARα activity | | |
| Fenofibrate | PPARα | Agonist, Hyperlipidemea drug | 500 | μM |
| Gemfibrozil | PPARα | Agonist, Hyperlipidemea drug | 163 | μM |
| Wyeth14643 | PPARα | Agonist; Potent peroxisome proliferators | 226 | μM |
| 15-Deoxy-Δ^{12,14}-Prostaglandin J2 | PPARγ | Agonist; Putative Natural ligand | 34 | μM |
| (PJ2) | | | | |
| MCC-555 | PPARγ | Agonist; Developmental insulin sensitizer; | 88 | μM |
| | | Unique Mechanism of Action | | |
| Ciglitazone (Cig) | PPARγ | Agonist; Hepatotoxic insulin sensitizer | 76 | μM |
| Troglitazone (Tro) | PPARγ | Agonist; Hepatotoxic insulin sensitizer | 18 | μM |
| GW-9662 | PPARγ | Antagonist | 125 | μM |
| 2-Bromohexadecanoic Acid (2BHDA) | PPARγ | Agonist; Synthetic Halogenated Fatty Acid | 94 | μM |
| Perfluourooctanoic Acid (PFOA) | PPARγ | Agonist; Synthetic Halogenated Fatty Acid | 191 | μM |

Treatment of HEPG2 cells with 2-Bromohydroxydecanoic acid, MCC-555, Ciglitazone, Trglitazone, Prostaglandin J2, PFOA, Gemfibrozil, Fenofibrate, Clofibrate, Bezafibrate, or Wyeth 14643 revealed a number of differentially expressed genes relative to a dimethylsulfoxide (DMSO) only treated control. Organization of the gene lists based on gene ontology classification indicated that four major functional groups or classifications of genes were identified from analysis of PPARγ ligands. As expected, a large number of differentially expressed genes for individual treatments were readily associated with PPARγ biology. Several other dominant themes were readily apparent from these data. In addition to the expected affects associated with PPARγ biology, changes in the expression of a large number of genes involved in cell growth (proliferation), programmed cell death (apoptosis), and the NFκB inflammatory response as a consequence of PPARγ ligand treatment were observed. These data suggested that there were significant differences in how different ligands for the same receptor affected each of these pathways. The data also suggests that there may be mechanistic relationships between NFκB activation and induction of apoptosis for several PPARγ ligands.

The effect of PPARγ ligands on induction of apoptosis in HepG2 cells was also examined. Compounds that affected the expression of a significant number of genes involved in apoptosis were potent inducers of apoptosis in the cell based assay. Moreover, the known hepatotoxic PPARγ ligands Ciglitazone and Troglitazone were potent inducers of apoptosis while PPARγ ligands that are safe and effective drugs did not induce apoptosis or only did so very modestly. The biomarkers identified in this screen as well as the cell based apoptotic induction assays can be used as surrogate assay screens to identify potentially toxic PPARγ drug candidates. In addition, these markers provide prognostic and diagnostic markers useful in disease diagnoses and patient stratification (Table 3).

TABLE 3
Therapeutic Areas and Chemical Classes with Demonstrated
Utility of the Methods of the Invention

| | Molecular | | |
|---|---|---|---|
| Therapeutic Area | Target | Chemical Class | Biology |
| Type 2 Diabetes | PPARγ | TZDs + Others | PPAR |
| | | | Inflammation |
| | | | Apoptosis |
| Dyslipidemia | PPARα | Fibrates + Others | PPAR |
| | | | Energy |
| | | | Homeostasis |
| | | | Mitogenic |
| High Cholesterol | HMG-CoA | Statins | Cataract toxicity |
| | Reductase | | Off target effects |
| Epilepsy | Unknown | Phenytoin | Teratogenic |
| | | | Cardiac interaction |
| | | | Off target effects |

Cell proliferation, apoptosis and the NFκB mediated inflammatory response are known to be intertwined through cross-talk of various signaling pathways and PPARγ signaling has been linked to each of these cellular processes. These pathways are critical to the utility of PPARγ ligands as anti-proliferative and anti-inflammatory agents but they must also be taken into account when evaluating the potential toxicities of PPARγ ligands, especially carcinogenicity and hepatotoxicity. Comparison of the effects of various PPARγ ligands on genes involved in proliferation, apoptosis and NFκB signaling indicated that different PPARγ ligands have distinct effects on these pathways. In particular, analysis of Ciglitazone and MCC-555 revealed that Ciglitazone had significantly more pronounced affects on gene expression relating to apoptosis and NFκB signaling. Analysis of Troglitazone data indicated that it too had marked differences in gene expression pertaining to these processes compared to other PPARγ ligands in the test set.

Apoptotic induction by PPARγ ligands has been reported in a wide range of cell types, including hepatocellular carcinomas (Yoshizawa, K., et al., Cancer, 95: 2243-2251 (2002); Shimada, T., et al., Gut, 50: 658-664 (2002); Lloyd, S., et al., Chem. Biol. Interact., 142: 57-71 (2002); Toyoda, M., et al., Gut, 50: 563-567 (2002)). This property has been suggested to contribute to the anti-proliferative activity of PPARγ ligands. However, the present invention is the first systematic investigation of the potency of a diverse set of PPARγ ligands in a common cell type. Moreover, the invention provides a demonstration that specific ligands of PPARγ that are safe and effective drugs nominally induce apoptosis in cells of hepatic lineage as well as a demonstration that known hepatotoxic PPARγ ligands are potent inducers of apoptosis. The cell-based apoptotic assay described herein can thus be used as a surrogate assay to discriminate hepatotoxic PPARγ ligands from compounds that are safe and non-hepatotoxic in humans.

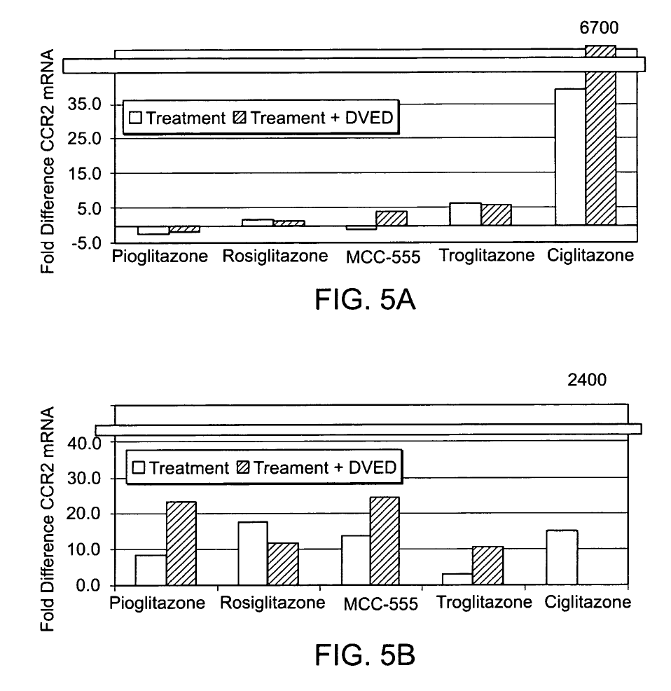

The microarray gene expression results indicated that there are underlying mechanistic differences in how PPARγ ligands produced distinct effects on NFκB signaling and apoptotic gene expression. PPARγ ligands are thought to achieve their anti-inflammatory activity, at least in part, via suppression of NFκB activity. Induction of apoptosis also elicits a strong anti-inflammatory response via suppression of NFκB activity. Based on these observations and the fact that Troglitazone and Ciglitazone were the only two known hepatotoxic TZDs in the test set, the potency of these compounds as well as several additional non-hepatotoxic TZDs was assessed in HepG2 cells. Indeed, Troglitazone and Ciglitazone were potent inducers of apoptosis. In contrast, Pioglitazone and Rosiglitazone did not induce apoptosis at all or only modestly did so. The developmental compound MCC-555 induced apoptosis intermediate to these two groups. To investigate the possibility that differential effects on apoptosis observed for various PPARγ ligands contributed to alteration of NFκB activity the effect of apoptosis inhibitors on the expression of target genes of NFκB when co-administered with PPARγ ligands was examined. The Caspase 3/7 inhibitor N-Acetly-Asp-Glu-Val-Asp-aldehyde (AC-DEVD-ACHO; DEVD) was used to block apoptosis and the effect of TZDs on the expression of pro-inflammatory chemokine/chemokine receptors known to be targets of NFκB was examined. It was hypothesized that if the suppression of NFκB activity arises from induction of apoptosis, then the inclusion of the DEVD should lead to an increase in expression of pro-inflammatory chemokines and chemokine receptors. Indeed, TZD-induced apoptosis in HepG2 cells was efficiently blocked using DEVD, indicating that induction of apoptosis was largely via a Caspase 3/Caspase 7 dependent pathway (FIG. 4). These data confirm the mechanistic indications of the microarray analysis and illustrate the utility of the “sentinel” strategy for predictive pharmacology and toxicology.

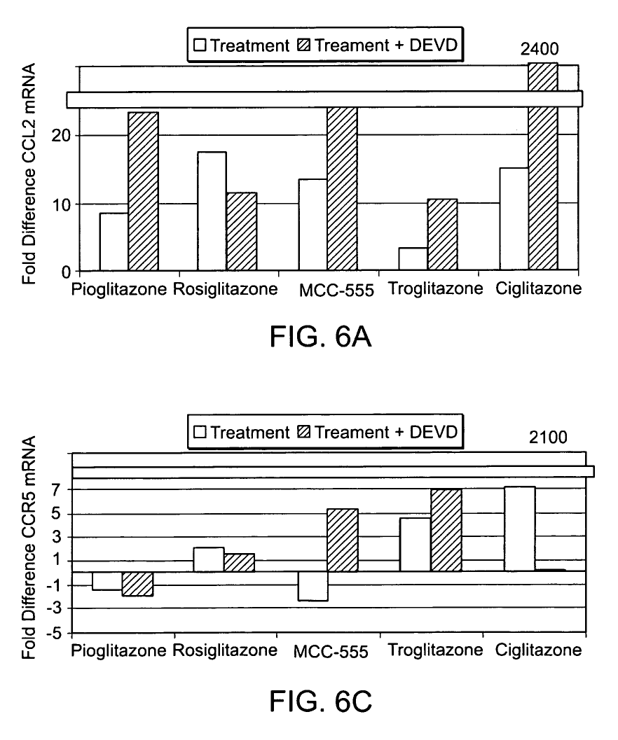

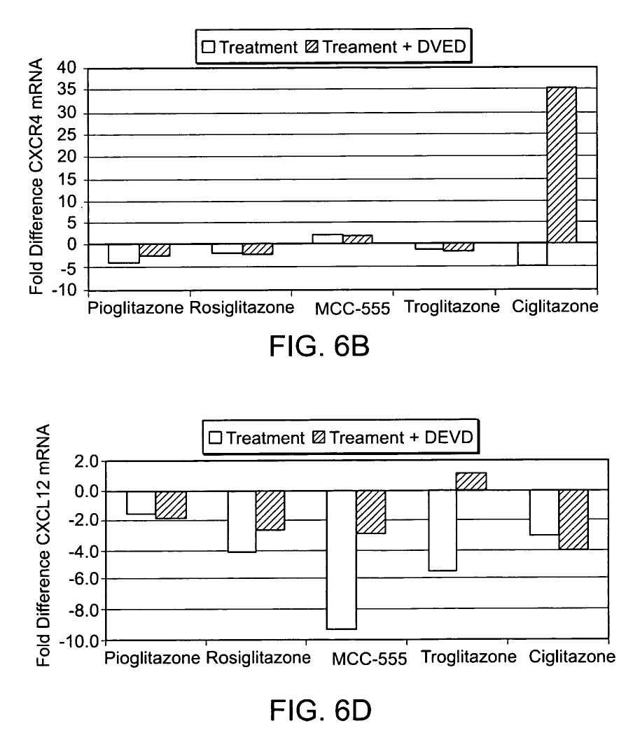

Chemokine Receptor 2 (CCR2) is the receptor of monocyte chemoattractant protein 1 (MCP-1; CCL2) which is a major inflammatory chemokine involved in arteriosclerosis and liver injury (Ishibashi, M., et al., Hypertension, 40: 687-693 (2002); Han, K. H., et al., J. Clin. Invest., 106: 793-802 (2000)). Quantitative Real Time Polymerase Chain Reaction (QRTPCR) was used to measure the relative abundance of CCR2 mRNA in cells treated with a TZD and cells treated with a TZD plus the Caspase 3/7 inhibitor DEVD. As can be seen in FIG. 5A, the expression of CCR2 in HepG2 cells relative to control is TZD dependent. Troglitazone and Ciglitazone LD₅₀ concentrations alone lead to 6× and 38× increased CCR2 expression, respectively. Under those conditions, Pioglitazone and MCC-555 suppressed CCR2 expression below that of control levels while Rosiglitazone does not significantly affect expression of CCR2. Inhibiting apoptosis in conjunction with treatment with Pioglitazone, MCC-555 or Troglitazone leads to an increase in CCR2 expression relative to compound only treatment, suggesting that apoptotic induction, even though it is undetectable in this assay for Pioglitazone, may contribute to the suppression of NFκB activity for these PPARγ ligands. In contrast, inhibition of apoptosis in conjunction with Rosiglitazone or Troglitazone treatment did not significantly affect CCR2 expression at these concentrations. The majority of these effects are dose dependent as seen in FIG. 5B, which illustrates the effects of equimolar treatments (175 μM). Treatment with Pioglitazone, Rosiglitazone or MCC-555 only leads to an increase in CCR2 mRNA relative to LD₅₀ concentrations. The values for Troglitazone and Ciglitazone are lower but this is likely due to the toxicity of these compounds which leads to significant cell death at 175 μM concentrations. The effects of inhibiting apoptosis are also recapitulated at the higher concentrations with the exception of Troglitazone.

The expression of several additional chemokines and chemokine receptors, including CCL2, CCR5, CXCL12, and CXCR4, were also examined in HepG2 cells at TZD concentrations equivalent to the LD₅₀ concentrations as well at an equimolar concentration (175 μM) (FIG. 5). Indeed, various PPARγ ligands have differing effects on the expression of several chemokines and chemokine receptors, including CCR2, CCL2, CXCL12, CCR5, and CXCR4, and inhibition of PPARγ ligand induced apoptosis led to increased expression of many of these mRNAs. These represent novel mechanistic findings and the cell based screens and the biomarkers identified through the methods of the invention analysis as well as other members of the indicated pathways represent useful tools to 1) screen for drug safer PPARγ drug candidates; 2) stratify responsive patient groups for clinical trials and 3) determine the safest medicine for specific patients in the clinic.

It is known that there is cross talk between the estrogen receptor (ER) and PPARγ but the mechanisms are not fully understood. Treatment of MCF7 cells with Estradiol causes the cells to grow. Treatment with Estradiol plus Rosiglitazone blocks proliferation. That is now believed as a consequence of ER and PPAR actions on CXCL12 expression. Thus, CXCL12 is at least one of the “cross roads” in this event. CXCR4 is also though to be involved, but the mechanism is unclear.

These data confirm that observations above that PPARγ ligands have differential effects on pro-inflammatory agent expression possibly as a result of differential induction of apoptosis that leads to suppression of NFκB activity. These observations suggest that PPARγ ligands can affect pro-inflammatory agent expression by distinct mechanisms and that some do so as a consequence of apoptotic induction and possibly via modulation of NFκB activity. These are novel observations with potential applications for 1) in vitro screening of developmental PPARγ ligands to eliminate potentially hepatotoxic compounds from development; 2) mechanistic biomarkers useful in discerning safe and effective anti-inflammatory mechanisms associated with PPARγ ligands; and 3) biomarkers useful for patient stratification in clinical trials and determining therapeutic courses involving PPARγ treatments.

In another embodiment, additional validation of the effects of PPARγ ligands on apoptosis and NFκB activation at the level of mRNA expression, protein expression, and pathway/cell based analysis can be performed. For example, a variety of cell lines including HepG2 cells, other transformed human cell lines of hepatic origin; primary human hepatocytes; transformed animal cell lines of hepatic origin as well as live animals can be used. Other cell and tissue types relevant to PPAR biology including pancreatic; muscle; adipose; endothelial; and immune systems, for example, can also be examined. The interconnections between apoptosis and proliferation indicate that the differential effects of PPARγ ligands demonstrated herein may play a role in the carcinogenicity potential of these agents. Thus, the relationship between proliferation, apoptosis and NFκB activity and their relevance to carcinogenicity of PPARγ ligands may also be examined.

In an embodiment, the array is a RiboChip, which affords several advantages over other gene expression platform. The RiboChip is predominantly (≧75%) comprised of features for detecting mRNAs for genes with a) known RNA binding domains (e.g. RNA recognition motif, K-homology domain, or pumillio domain), b) known RNA binding function (e.g. ACO1), c) functions associated with RNA metabolism (e.g. RNA splicing, RNA editing, or RNA degradation); and d) RNA synthesis (e.g., transcription). The remaining features represent genes associated with nuclear receptors, nuclear receptor co-activators, and nuclear receptor co-repressors. The inclusion of the latter group of features is based on emerging evidence that many of the proteins encoded by these genes possess RNA binding capability. The size of the data sets are generally smaller and therefore easier to manage, analyze and interpret. The gene content is readily linked to biological pathways and processes. The segregation of regulatory genes from the bulk of other genes in the human gene potentially enables more reliable detection of modest changes in gene expression as well as low abundance transcripts. Methods of the invention include those disclosed in U.S. Pat. No. 6,635,422.

Practice of the invention will be still more fully understood from the following examples, which are presented herein for illustration only and should not be construed as limiting the invention in any way.

### EXEMPLIFICATION

### Example 1

### Determining the Cytotoxicity of Test Compounds

HepG2 cells were obtained from American Type Culture Collection (ATCC, Manassas, Va., cat. no. HB-8065). Cells were maintained as recommended in Minimal Essential Medium (MEM) (Gibco-BRL, a Division of Invitrogen, Carlsbad, Calif.) with 10% fetal bovine serum (FBS, HyClone, Logan, Utah) supplemented with antibiotics in p150 plates at 37° C., 5% CO₂. Cells were split 1:5 and fresh media added every 3 days.

Cytotoxicity was assessed using the Alamar Blue-based CellTiter™ Blue Cell Viability Assay (Promega, Madison, Wis.) to determine the viable cell fraction that remained following a 72 hour treatment period. Cells (˜8,000 cells/well) were plated in 96 well BioCoat collagen coated plates (Becton Dickinson, Franklin Lakes, N.J.) using standard media. This allowed untreated control samples (0.25% DMSO) to be in late log phase (˜70% confluent) at completion of the study. Cells were then allowed to recover for 24 hours at 37° C., 5% CO₂. A two (2) fold dilution series was prepared for each compound starting at 3.0 mM in MEM containing 0.1% BSA (instead of 10% FBS) but without phenol red or antibiotics. Following the cell recovery period, the media was removed and fresh media containing compound was added. Treatments were performed in triplicate for each compound at each dose. Cells were incubated with compound for 72 hours at 37° C., 5% CO₂. The viable cell fraction remaining was determined by washing the wells with fresh media without indicator, lysing the remaining live cells by adding 0.9% Triton X-100 (Sigma, St. Louis, Mo.) in water, and performing the Alamar Blue assay as described in the CellTiter™ Blue Cell Viability Assay product literature. The concentration resulting in 50% cell death relative to a vehicle only control (0.25% final DMSO) following 72 hours of treatment with a compound (LD₅₀) was determined using Prism 4.0 (GraphPad, San Diego, Calif.) dose-response analysis.

### Example 2

### Determining the Apoptosis in Response to Test Compounds

Apoptosis was assessed using the Apo-OneR Homogeneous Caspase-3/7 Assay (Promega) to determine the activity of an early apoptotic event: Caspase 3/7 activation. Cells (˜40,000 cells/well) were plated in 96 well plates (Corning, Acton, Mass., cat. no. 3595) using plating media (MEM, 1× Sodium Pyruvate, 1× NEAA, 10% FBS). Cells were then allowed to grow for 24 hours at 37° C., 5% CO₂, and then serum starved by changing to serum free media (MEM, 1× Sodium Pyruvate, 1× NEAA, 0.1% BSA). Cells were allowed to remain in the serum free media for a further 24 hours. At 48 hours post-plating the media was removed and replaced with a test compound diluted in serum free media. A dilution series was created for each compound through serial dilutions performed in a separate plate and later transferred to the cells. Initially, a broad dilution series was conducted from ˜300 μM to ˜1 μM to determine approximate maximum tolerated and minimum effective concentrations. Based on these initial dose response studies, refined dilution series were performed for each compound to obtain dose response curves with at least 2 data points (concentrations) defining the unaffected (0% apoptosis) and maximally affected concentrations. Treatments were performed in quadruplicate for each compound at each dilution. If the Caspase 3/7 inhibitor AC-DEVD-CHO (DEVD) was used it was mixed with the compound prior to the addition to the cells. DMSO was kept constant at 0.1% in compound-only experiments and 0.2% with inhibitor experiments. Cells were incubated with compound for 24 hours at 37° C., 5% CO₂. The level of apoptosis was determined by adding the caspase 3/7 substrate Z-DEVD-Rhodamine110, dissolved in buffer supplied by the manufacturer, to each well. The plate was incubated at room temperature for 1 hour. The media and buffer/substrate mixture was removed and placed in a Corning 96 well black walled plate (Corning, cat. no.3651) and read on a fluorescent plate reader at excitation: 485±20 and emission: 530±25. Additionally the plate was further incubated overnight at room temperature for slightly higher relative fluorescence units (RFUs). The amount of Caspase 3/7 activity was compared to a vehicle only control.

### Example 3

### Preparation of RNA

RNA for microarray analysis was obtained from cells treated for 24 hours at the determined LD₅₀. Typically, ˜1.5×10⁶ cells were plated in a p100 dish and allowed to settle for 24 hours by incubation at 37° C., 5% CO₂ in MEM+10% FBS without antibiotics. Old media was removed and fresh MEM+0.1% BSA without antibiotics containing a test compound at LD₅₀ concentration and 0.25% DMSO was added to the flask. A vehicle-only treatment was also performed. Duplicate treatments were performed for each compound as well as for vehicle-only controls. The cells were incubated with compound for 24 hours at 37° C., 5% CO₂ and were harvested by scraping (without trypsinization) and centrifugation. The cell pellets were flash frozen and stored at −80° C. until ready for RNA extraction.

Total RNA was isolated using RNeasy Midi or Maxi kits (Qiagen) according to methods described by the manufacturer. Total RNA (100 μg) was routinely treated with 40 Units DNaseI (Ambiom, cat.#2222) in a total volume of 450 mL 1× DNaseI buffer at 37° C. for 20-30 minutes to remove contaminating DNA. The reaction was stopped by extraction with acid phenol/chloroform/isoamyl alcohol (25:24:1) (Sigma, St. Louis, Mo.). The RNA was precipitated by transferring the aqueous layer to a clean tube; adjusting to ˜2.5 M ammonium acetate (⅓ volume 7.5 M stock); incubating at −80° C. of ≧20 minutes, and centrifugation at ˜18,000 g for 20 minutes, 4° C. The pelleted RNA was rinsed with 70% ethanol and allowed to air dry. Purified, Dnase I treated RNA was routinely analyzed using an Agilent 2100 Bioanalyzer (Agilent, Palo Alto, Calif.). RNA was assessed for purity by examining electropherograms for the presence of broad peaks overlapping the 28S and 18S ribosomal RNA (rRNA) peaks. Broad peaks of this nature indicate contamination with genomic DNA. If such contamination was detected, the RNA was retreated with DNase I and purified as described above. In addition, the relative abundance of 28S to 18S rRNA was determined to assess the quality of the RNA sample. Ratios greater than or equal to about 1.7 for 28S/18S rRNA indicate little or no degradation of the RNA and are acceptable for microarray analysis. Ratios less than about 1.7 indicate degraded RNA that is not acceptable for microarray analysis.

### Example 4

### Screening the Microarray

Aminoallyl cDNA was synthesized based on modifications of protocols by DeRisi (www.microarray.org; “Reverse Transcription and aa-UTP Labeling of RNA”) and TIGR (www.tigr.org; Protocol M005). Briefly, total RNA (10 μg) was combined with 2 μl dT₁₈ (200 μM), 2 μl random decamer (1 mM stock), and diethyl pyrocarbonate (DEPC) treated water to a final volume of 17.5 μl. Primers were annealed to the RNA template by heating at 70° C. for 10 minutes and then cooling to room temperature or on ice. Aminoallyl cDNA was synthesized by addition of combining the above reaction with 6 μl SuperScript II first strand buffer, 3 ml 0.1 M dithiothreitol, 0.6 ml 50× labeling mix (25 mM dATP, 25 mM dGTP, 25 mM dCTP, 15 mM dTTP, and 10 mM aminoallyl-dUTP (Sigma; St. Louis, Mo.; Catalog A0410)), 1 ml RNAseOUT (Invitrogen; Carlsbad, Calif.; Catalog 10777-019), and 1 ml SuperScript II (Invitrogen; Carlsbad, Calif.; Catalog 18064-022) followed by incubation for 3 to 24 hours at 42° C. The RNA was hydrolyzed by addition of 10 μl each 1 M NaOH and 0.5 M ethylenediamine tetraacetic acid followed by incubation for 15 minutes at 65° C. The solution was neutralized by addition of 10 μl of 1 M HCl. The aminoallyl-cDNA was purified using a QiaQuick PCR purification kit (Qiagen) with the following modifications. The cDNA was mixed with 5× reaction volumes of the Qiagen supplied PB buffer and transferred to a QIAquick column. The column was placed in a collection tube and centrifuged for 1 minute at 13,000 rpm. The column was washed by addition of 750 μl of phosphate wash buffer (prepared by mixing 0.5 mL 1 M KPO₄ (9.5 mL 1M K₂HPO₄+0.5 mL 1M KH₂PO₄), pH 8.5; 15.25 RNase free water; and 84.25 mL 95% ethanol) and centrifuging at 13,000 rpm. The wash step was repeated and the column centrifuged 1 minute at maximum speed to remove all traces of wash solution. The column was transferred to a clean collection tube and the aa-cDNA was eluted by addition of 30 μl of phosphate elution buffer (prepared by mixing 0.5 mL 1 M KPO_{4,} pH 8.5; 15.25 RNase free water; and 84.25 mL 95% ethanol). The elution was repeated once and the sample was dried in a speed-vac.

Coupling of Cyanin Reactive Esters to aa-CDNA and Purification of Labeled cDNA

The purified aa-cDNA was coupled to cyanine dyes (Amersham Biosciences; Piscataway, N.J.; Catalog # PA23001 (Cy-3) or PA25001 (Cy5)); purified; and analyzed as described. Stock solutions of Cyanin3 and Cyanin5 reactive N-hydroxysuccinamide dye were prepared by dissolving one tube of reactive dye in 73 μl of anhydrous DMSO. Reactive dye was coupled to aa-cDNA by addition of 4.5 μl reactive DMSO dye solution to the aa-cDNA and incubating for 1 hour in the dark at room temperature. Following coupling, the dye-labeled cDNA was purified using standard QIAquick PCR cleanup kit methods and buffers. The labeling reactions were analyzed for incorporation according The Institute for Genomic Research labeling protocol, TIGR M005.

Hybridization and Processing of Spotted Microarrays

Each spotted microarray is sufficient for analysis of two Cy-dye labeled samples, one labeled with Cy3 and one labeled with Cy5. For each microarray, material from one Cy3 labeling and one Cy5 labeling reaction were pooled and dried in a speed vac. The pooled samples were then hybridized to the microarray and the slides processed according to the general guidelines suggested by the manufacturer (MWG Biotech; High Point, N.C.).

Microarray Data Extraction and Analysis

Microarrays were scanned using an Axon 4000B Scanner and GenePix version 4.0 software (Axon; Union City, Calif.). The resulting image files were quantified using BioDiscovery's Imagene software version 4.2 (El Segundo, Calif.) using standard background and spot finding settings. The complete microarray study was conducted as a closed loop-design with a set of 6 nested loops each containing a common reference sample. Processed slides were scanned using an Axon GenePix 4000b scanner and GenePix Pro software v 4.0 (Axon, Union City, Calif.). Intensity data was extracted from TIFF images using Imagene v 4.2 (BioDiscovery, El Segundo, Calif.). Custom applications were developed to import the intensity data into the R statistical environment v 1.7.1 (www.r-project.org) and the BioConductor micrarray libraries v 1.2 (www.bioconductor.org). Data preprocessing, including background subtraction, Lowess scale and location normalization, flooring and quality control analysis, was conducted using standard BioConductor functions. Prior to extracting the ciglitazone, MCC-555 and DMSO data subsets, the MAD function of BioConductor was applied to achieve between-slide scale normalization. This step was included to facilitate analysis of the ciglitazone, MCC-555 and DMSO sections of the experiment as single channel data sets. This significantly simplified visualization and analysis of the differential expression for these treatments. The validity of this approach was determined by comparing differential expression results determined using MAANOVA, which is specifically developed for analysis of loop designs, and using ANOVA analysis (see below) as well as by comparing class prediction results on raw and single channel data. The results were substantially the same indicating that analysis of the scaled data as single channel measurements was a valid strategy.

The preprocessed data for Ciglitazone, MCC-555 and DMSO were exported from R and then imported into GeneSpring v 6.1 (Silicon Genetics, Redwood City, Calif.) for differential expression analysis and clustering. Flooring as well as between gene and between-channel median scaling was applied to the data. Differential expression was determined using the ANOVA (Welch's t-test) parametric test assuming unequal variance, p≦0.05 and using the cross-gene error model to account for between chip variations. No false discovery rate correction could be applied due to only 4 replicates (2 biological replicates each analyzed by dye swap) being available for each treatment. GeneSpring was also used for K-means and QT clustering using the standard correlation function of the software as well as for class prediction analyses (data not shown).

### Example 5

### Quantitative Real Time PCR

Quantitative Real Time PCR was conducted using a BioRad iCycler iQ with iCylcler software v 3.0.6070 (Biorad, Hercules, Calif.). Total RNA was prepared and verified for integrity as described above for microarray analysis. First strand cDNA syntheses were conducted using Superscript II (Invitrogen; Carlsbad, Calif.; cat. no. 10777-019) as described by the manufacturer using 125 ng random decamer primer per 1 μg of total RNA. The RNA was distributed into a 96 well RT-QPCR plate at 10-50 ng/well. Real time quantitation was performed using IQ Syber Green Supermix (BioRad, cat. no. 170-8882) per the manufacturer's recommendations. A step amplification protocol was used incorporating a 30 second 95° C. denaturation step and a 60 second 60° C. amplifaction step. The Delta-Delta CT method (Applied BioSystems User Bulletin 2, Foster City, Calif.) was used to calculate relative mRNA abundance using 18s rRNA as the internal reference. Gene specific primers were used and are shown are shown below.

TABLE 4

Gene-Specific Primers Used for Quantitative PCR Analysis

| | Representative | | |
|---|---|---|---|
| RNA | GenBank ID | Primer 1 | Primer 2 |
| 18s rRNA | X03205 | CCATCCAATCGGTAGTAGCG | GTAACCCGTTGAACCCCATT |
| CCR2 | NM_000647 & | CGGTGCTCCCTGTCATAAAT | TGAACACCAGCGAGTAGAGC |
| | NM_000648 | | |
| CCL2 | NM_002982 | CCCAAACTGCGAAGACTTGA | GGGGAAAGCTAGGGGAAAAT |
| CXCR4 | NM_003467 & | GGCCCTAGCTTTCTTCCACT | GGGCAGAGGTTTTAAATTTGG |
| | NM_001008540 | | |
| CCR5 | NM_000579 | CGTGTCTCCCAGGAATCATC | TGAGAGCTGCAGGTGTAATGA |

### Example 6

### Differential Expression Analysis of MCC-555 and Ciglitazone

To gain insight into the similarities and differences of the pharmacology and toxicology for MCC-555 and ciglitazone, a series of statistical analyses were conducted on the MCC-555, ciglitazone and DMSO data sets to identify genes that were affected by one or both compounds. Genes affected by both compounds represent candidate markers for common pharmacological and toxicological effects and genes that are uniquely affected by one compound are likely markers for distinct pharmacological and toxicological properties. Differentially expressed genes were identified using the Analysis of Variance (ANOVA; Welch's t-test assuming unequal variance) function of GeneSpring. ANOVA analysis (p ≦0.05) revealed 33 and 93 genes were differentially expressed for MCC-555 and ciglitazone treatments, respectively. An additional ANOVA analysis was conducted to directly determine differences in expression between MCC-555 and ciglitazone. This identified 48 genes that were differentially expressed (p-value ≦0.05) between the ciglitazone and MCC-555 data sets, 21 of which were not identified by the other ANOVAs. The three gene lists were pooled to provide a master list of 146 differentially expressed genes. This master gene list was sorted based on similarities and differences in expression for the MCC-555 and ciglitazone treatments relative to the DMSO control and were segregated based on relative expression.

Some genes were up-regulated by both treatments and some genes were down-regulated by both treatments. Functional classification of the gene lists was initially performed using GoMiner (Zeeberg, B. R., et al., Genome Biol., 4: R28( 2003)). Gene Ontology (GO), a hierarchical and structured classification of gene/protein function, is the basis of the GoMiner classification. Each gene is further annotated based on gene specific functional information and subdivided based on the major biological processes associated with the gene lists. Functions included cell growth/apoptosis (development, proliferation, apoptosis, G1 arrest, PPAR activity, NFκB activity, differentiation, mitochondrial biogenesis, translation, nephrosis); stress/inflammation (including interferon response, inflammation); trafficking (including vesiculation, glycoprotein trafficking receptors, mRNA trafficking, protein trafficking, protein folding, exocytosis, multidrug resistance); macromolecular mechanisms (translation, transcription, iron homeostasis, RNA splicing, RNA metabolism, mRNA processing, splicing, synaptic signaling, mitochondrial, steroidogenesis, respiration, translational suppression, gene silencing); and other. The genes within these major divisions were sorted based on MCC-555 differential expression (DE). The fold DE for ciglitazone and MCC-555 treatments relative to the DMSO control as well as the CV value for each DE value was determined. The genes within each primary functional classification were ordered based on MCC-555 DE values.

Genes affected differently by MCC-555 and ciglitazone were also determined, including genes only affected by MCC-555, genes only affected by ciglitazone, and genes whose expression was affected in opposing directions for the MCC-555 and ciglitazone treatments. Initial functional classification was performed using automated GO annotation using GoMiner. Additional processes and functions associated with each gene were determined and subdivided into major biological processes associated with the genes and the genes within each major subdivision were sorted according to MCC-555 DE values. The additional or extended functions were also determined. The fold differential expression for ciglitazone and MCC-555 treatments relative to the DMSO control as well as the CV value for each DE value was also determined.

### INCORPORATION BY REFERENCE

The contents of all cited references (including literature references, patents, patent applications, and websites) that maybe cited throughout this application are hereby expressly incorporated by reference. The practice of the present invention will employ, unless otherwise indicated, conventional techniques and materials of molecular biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

We claim:

1. An ex vivo method for predicting and/or determining a certain pharmacological and/or toxicological effect of a compound in vivo, the method comprising the steps of:
(a) treating a cell with a compound;
(b) preparing RNA from the treated cell;
(c) hybridizing the RNA to a microarray consisting essentially of a plurality of nucleic acids that encode regulators of gene expression and modulators of biological pathways and/or processes involved in pharmacology and toxicology; and
(d) identifying altered gene expression of the regulators and/or modulators, wherein the altered gene expression is indicative that administration of the compound will have a certain pharmacological and/or toxicological effect in vivo.

2. An ex vivo method for predicting and/or determining a certain pharmacological and/or toxicological effect of a receptor ligand in vivo, the method comprising the steps of:
(a) treating a cell with a receptor ligand;
(b) preparing RNA from the treated cell;
(c) hybridizing the RNA to a microarray comprising a plurality of nucleic acids that encode regulators of gene expression and modulators of biological pathways and/or processes involved in pharmacology and toxicology; and
(d) identifying altered gene expression of the regulators and/or modulators, wherein the altered gene expression is indicative that administration of the receptor ligand will have a certain pharmacological and/or toxicological effect in vivo.

3. An ex vivo method for identifying a safe drug candidate, the method comprising the steps of:
(a) treating a cell with a compound;
(b) preparing RNA from the treated cell;
(c) hybridizing the RNA to a microarray comprising a plurality of nucleic acids that encode regulators of gene expression and modulators of biological pathways and/or processes;
(d) identifying altered gene expression of the regulators and/or modulators, wherein the altered gene expression is indicative that administration of the compound will have a certain pharmacological and/or toxicological effect in vivo; and
(e) determining the ability of the compound to induce apoptosis and/or cell death in the cell.

4. An ex vivo method for identifying one or more biomarkers for an altered biological pathway(s) and/or process(es) in a cell that has been treated with a compound, the method comprising the steps of:
(a) treating a cell with a compound;
(b) preparing RNA from the treated cell;
(c) hybridizing the RNA to a microarray comprising a plurality of nucleic acids that encode regulators of gene expression and modulators of biological pathways and processes; and
(d) identifying altered gene expression of the regulators and/or modulators, wherein the regulators and/or modulators with altered gene expression are biomarkers for an altered biological pathway(s) and/or process(es) that involves the regulators and/or modulators.

5. An ex vivo method for identifying one or more biomarkers indicative of a certain toxic effect of a compound, the method comprising the steps of:
(a) treating a cell with a compound that has a certain toxic effect;
(b) preparing RNA from the cell;
(c) hybridizing the RNA to a microarray comprising a plurality of nucleic acids that encode regulators of gene expression and modulators of biological pathways and/or processes involved in toxicity; and
(d) identifying altered gene expression of the regulators and/or modulators, wherein the altered gene expression is indicative of a certain toxic effect of the compound in vivo.

6. An ex vivo method for identifying a biological pathway(s) and/or process(es) that is altered in response to treating a cell with a compound, the method comprising the steps of:
(a) treating a cell with a compound;
(b) preparing RNA from the treated cell;
(c) hybridizing the RNA to a microarray comprising a plurality of nucleic acids that encode regulators of gene expression and modulators of biological pathways and/or processes; and
(d) identifying altered gene expression of the regulators and/or modulators, wherein the altered gene expression is indicative that the compound acts via the biological pathway(s) and/or process(es) that involves the regulators and/or modulators.

7. An ex vivo method for identifying a functional relationship between at least two biological pathways and/or processes in a cell in response to treatment with a compound, the method comprising the steps of:
(a) treating a cell with a compound;
(b) preparing RNA from the treated cell;
(c) hybridizing the RNA to a microarray comprising a plurality of nucleic acids that encode regulators of gene expression and modulators of biological pathways and/or processes; and
(d) identifying altered gene expression of the regulators and/or modulators, wherein the altered gene expression of regulators and/or modulators that participate in different biological pathways and/or processes is indicative that there is a functional relationship between the biological pathways and/or processes in response to the compound.

8. The method according to claim 7, wherein the pathways comprise an apoptotic pathway and an NFκB pathway.

9. The method according to claim 7, wherein the pathways comprise an apoptotic pathway and an inflammatory response pathway.

10. The method according to claim 1 or 7, wherein the pathway comprises a cell death pathway.

11. The method according to claim 1, the method further comprising the step of comparing the altered gene expression of the regulators and/or the modulators in response to the compound to the altered gene expression caused by a treatment with another compound.

12. The method according to claim 1, the method further comprising the step of determining the level of cell death in response to treatment with the compound.

13. The method according to claim 1, the method further comprising the step of determining the level of apoptosis in the treated cell.

14. The method according to claim 1, wherein the regulator or modulator is selected from the group consisting of a factor that regulates transcription, a factor that regulates post-transcriptional gene expression, a factor that regulates a pharmacological pathway and/or process, and a factor that regulates a toxocological pathway and/or process.

15. The method according to claim 1, wherein the regulator or modulator having altered gene expression is a pro-inflammatory factor.

16. The method according to claim 1, wherein the regulator or modulator having altered gene expression is an anti-inflammatory factor.

17. The method according to claim 1, wherein the regulator or modulator having altered gene expression is selected from the group consisting of CCR2, CCL2, CCR5, CXCR4, and CXCL12.

18. The method according to claim 1, wherein the regulator or modulator having altered gene expression is CXCL12.

19. The method according to claim 7, wherein the method uncouples the effects of the compound on two or more pathways.

20. The method according to claim 19, wherein the pathways comprise an efficacy pathway and a toxicity pathway.

21. The method according to claim 19, wherein the pathways comprise a PPAR efficacy pathway and a PPAR toxicity pathway.

22. The method according to claim 1, wherein the regulator or modulator having altered gene expression is involved in apoptosis.

23. The method according to claim 1, wherein the regulator or modulator having altered gene expression is involved in the inflammatory response.

24. The method according to claim 1, wherein the regulator or modulator having altered gene expression is involved in lipid metabolism.

25. The method according to claim 1, wherein the regulator or modulator having altered gene expression is involved in cellular maturation or cellular differentiation.

26. The method according to claim 25, wherein the regulator or modulator having altered gene expression is involved in the cellular maturation or differentiation of adipocytes.

27. The method according to claim 1, wherein the regulator or modulator having altered gene expression is involved in lipogenesis.

28. The method according to claim 1, wherein the regulator or modulator having altered gene expression is involved in carcinogenicity.

29. The method according to claim 1, wherein the altered gene expression is a biomarker for breast cancer.

30. The method according to claim 1, wherein the regulator or modulator having altered gene expression is involved in glucose metabolism.

31. The method according to claim 1, wherein the regulator or modulator having altered gene expression is involved in cell proliferation.

32. The method according to claim 1, wherein the regulator or modulator having altered gene expression is involved in edema.

33. The method according to claim 1, wherein the biological pathway and/or process is selected from the group consisting of a cellular pathway or process, a physiological pathway or process, a biochemical pathway or process, a metabolic pathway or process, and a signaling pathway or process.

34. The method according to claim 4, wherein the biomarker is involved in a pathway or process selected from the group consisting of the inflammatory response, apoptosis, NFκB signaling, lipid metabolism, cellular maturation, cellular differentiation, lipogenesis, carcinogenicity, glucose metabolism, PPAR signaling, cell proliferation, and edema.

35. The method according to claim 34, wherein the regulator or modulator having altered gene expression is involved in the cellular maturation or differentiation of adipocytes.

36. The method according to claim 1, wherein the pharmacological or toxicological effect is apoptosis.

37. The method according to claim 1, wherein the pharmacological or toxicological effect is cell growth.

38. The method according to claim 1, wherein the pharmacological or the toxicological pathway acts at least in part via a ligand activated nuclear hormone receptor.

39. The method according to claim 1, wherein the pharmacological or the toxicological pathway acts via an estrogen receptor.

40. The method according to claim 1, wherein the pharmacological or the toxicological pathway acts via a receptor selected from the group consisting of NR2F1, NR5A2, NR2E3, NR4A2, NR0B1, NR3C1, NR4A3, NR2C2, NR1D1, NR2F2, NR3C2, NR1I2, NR1D2, NC2C1, NR2E1, NR4A1, NR1H3, NR1H4, NR1I3, NR6A1, NR1H2, NR5A1, RARA, RARB, RARG, THRB, THRA, ESRRB, ESR2, ESRRA, ESRRG, ESR1, HNF4G, HNF4A, PPARG, PPARA, PPARD, PGR, VDR, RXRA, RXRG, RORB, RORC, RORA, GRLF1, FOXA1, and NCOA5.

41. The method according to claim 1, wherein the identifying step comprises comparing gene expression of the treated cell to gene expression of control cell.

42. The method according to claim 40, wherein the control cell is an untreated cell.

43. The method according to claim 40, wherein the control cell is a cell that is treated with a toxic compound.

44. The method according to claim 40, wherein the control cell is a cell that is treated with a non-toxic compound.

45. The method according to claim 1, wherein the cell is a cultured cell.

46. The method according to claim 1, wherein the cell is a hepatic cell.

47. The method according to claim 1, wherein the cell is a hepatocellular carcinoma.

48. The method according to claim 1, wherein the cell is a HEPG2 cell.

49. The method according to claim 1, wherein the cell is selected from the group consisting of a primary hepatocyte, a primary non-human hepatocyte, a transformed animal cell, a hepatic cell in a live animal, a pancreatic cell, a muscle cell, an adipose cell, breast cell, kidney cell, and an endothelial cell.

50. The method according to claim 1, wherein the cell is an immune cell.

51. The method according to claim 1, wherein the cell is an Kupffer cell.

52. The method according to claim 1, wherein the compound is a nuclear receptor ligand.

53. The method according to claim 1, wherein the compound is an estrogen receptor ligand.

54. The method according to claim 1, wherein the compound is a peroxisome proliferator activated receptor ligand.

55. The method according to claim 1, wherein the compound is a peroxisome proliferator activated receptor gamma (PPARγ) ligand.

56. The method according to claim 1, wherein the compound is a peroxisome proliferator activated receptor alpha (PPARα) ligand.

57. The method according to claim 1, wherein the compound is a peroxisome proliferator activated receptor delta (PPARδ) ligand.

58. The method according to claim 1, wherein the compound is selected from the group consisting of pioglitazone, rosiglitazone, MCC-555, troglitazone, ciglitazone, 2-bromohydroxydecanoic acid, prostaglandin J2, PFOA, gemfibrozil, fenofibrate, clofibrate, benzafibrate, and Wyeth 14623.

59. The method according to claim 1, wherein the method detects the activation of NFκB as a consequence of PPAR apoptosis.

60. The method according to claim 1, wherein the toxicity comprises hepatotoxicity.

61. The method according to claim 1, wherein the altered gene expression is indicative of a safe and effective anti-inflammatory mechanism associated with a peroxisome proliferator activated receptor ligand.

62. The method according to claim 1, wherein the altered gene expression is indicative of the safety of a therapeutic treatment comprising the compound.

63. The method according to claim 1, wherein the altered gene expression is indicative of the carcinogenicity of the compound.

64. The method according to claim 1, wherein the altered gene expression is useful for grouping or stratifying a patient population according to which regulators or modulators had altered gene expression in response to the compound.

65. The method according to claim 1, wherein the patient population is participating in a clinical trial.

66. The method according to claim 1, wherein the cell is treated with an LD₅₀ dose of the compound.

67. The method according to claim 1, wherein the cell is treated with a dose of the compound that is lower than the LD₅₀ dose.

68. The method according to claim 1, wherein the compound is known or suspected to exert an effect on gene expression via a peroxisome proliferator activated receptor.

69. The method according to claim 1, wherein the cell is treated for 24 hours with an LD₅₀ dose.

70. The method according to claim 1, wherein the cell is treated for about 2, about 4, about 6, about 8, about 10, about 12, about 14, about 16, about 18, about 20, or about 22 hours.

71. The method according to claim 1, wherein the gene expression of a gene that regulates cell growth is altered.

72. The method according to claim 1, wherein the gene expression of a gene that regulates apoptosis is altered.

73. The method according to claim 1, wherein the gene expression of a gene that regulates an inflammatory response is altered.

74. The method according to claim 76, wherein the inflammatory response is mediated by NFκB.

75. The method according to claim 1, wherein the pathway comprises a nuclear receptor activation pathway.

76. The method according to claim 1, wherein the pathway comprises an NFκB activation pathway.

77. The method according to claim 1, wherein the regulator or modulator participates in a pathway or process selected from the group consisting of cell growth, cell proliferation, cell development, cell differentiation, apoptosis, stress, inflammation, trafficking, macromolecular metabolism, RNA splicing, mRNA metabolism, transcription, translation, protein folding, exocytosis, multidrug resistance, respiration, iron homeostasis, and cholesterol homeostasis.


SEQUENCE LISTING (11 sequences)

SEQ ID NO: 1 - protein, 4 aa, Artificial Sequence
  Description of Artificial Sequence: Synthetic peptide
DEVD

SEQ ID NO: 2 - DNA, 20 bp, Artificial Sequence
  Description of Artificial Sequence: Synthetic primer
ccatccaatcggtagtagcg

SEQ ID NO: 3 - DNA, 20 bp, Artificial Sequence
  Description of Artificial Sequence: Synthetic primer
gtaacccgttgaaccccatt

SEQ ID NO: 4 - DNA, 20 bp, Artificial Sequence
  Description of Artificial Sequence: Synthetic primer
cggtgctccctgtcataaat

SEQ ID NO: 5 - DNA, 20 bp, Artificial Sequence
  Description of Artificial Sequence: Synthetic primer
tgaacaccagcgagtagagc

SEQ ID NO: 6 - DNA, 20 bp, Artificial Sequence
  Description of Artificial Sequence: Synthetic primer
cccaaactccgaagacttga

SEQ ID NO: 7 - DNA, 20 bp, Artificial Sequence
  Description of Artificial Sequence: Synthetic primer
ggggaaagctaggggaaaat

SEQ ID NO: 8 - DNA, 20 bp, Artificial Sequence
  Description of Artificial Sequence: Synthetic primer
ggccctagctttcttccact

SEQ ID NO: 9 - DNA, 21 bp, Artificial Sequence
  Description of Artificial Sequence: Synthetic primer
gggcagaggttttaaatttgg

SEQ ID NO: 10 - DNA, 20 bp, Artificial Sequence
  Description of Artificial Sequence: Synthetic primer
cgtctctcccaggaatcatc

SEQ ID NO: 11 - DNA, 21 bp, Artificial Sequence
  Description of Artificial Sequence: Synthetic primer
tgagagctgcaggtgtaatga